SEQUENCE LISTING (51 sequences)

SEQ ID NO: 1 - RNA, 19 nt, Artificial Sequence
  siRNA
gacaggagcuacaaaguaa

SEQ ID NO: 2 - DNA, 19 bp, Artificial Sequence
  siRNA
ccaugaauaucacagcuua

SEQ ID NO: 3 - DNA, 19 bp, Artificial Sequence
  siRNA
ccgaagagcuugacuacuu

SEQ ID NO: 4 - DNA, 19 bp, Artificial Sequence
  siRNA
ccacgugucugcccaauua

SEQ ID NO: 5 - DNA, 21 bp, Artificial Sequence
  siRNA
gaggttggagtgccaatagaa

SEQ ID NO: 6 - DNA, 21 bp, Artificial Sequence
  siRNA
ggacaggttagtctgaatgat

SEQ ID NO: 7 - DNA, 21 bp, Artificial Sequence
  siRNA
ggacaacaagcggtacatctt

SEQ ID NO: 8 - DNA, 21 bp, Artificial Sequence
  siRNA
gcaagacaaacgtgtacatct

SEQ ID NO: 9 - DNA, 21 bp, Artificial Sequence
  siRNA
gtggagacatgagcgatgaga

SEQ ID NO: 10 - DNA, 21 bp, Artificial Sequence
  siRNA
ggagacatgagcgatgagaga

SEQ ID NO: 11 - DNA, 21 bp, Artificial Sequence
  siRNA
gccaactgtaccgccatctat

SEQ ID NO: 12 - DNA, 21 bp, Artificial Sequence
  siRNA
ggaaactggagcgattctaaa

SEQ ID NO: 13 - DNA, 21 bp, Artificial Sequence
  siRNA
gaaactggagcgattctaaat

SEQ ID NO: 14 - DNA, 21 bp, Artificial Sequence
  siRNA
ggcaatctgacagctcataca

SEQ ID NO: 15 - DNA, 21 bp, Artificial Sequence
  siRNA
gtcagtaaggatgagcagtat

SEQ ID NO: 16 - DNA, 21 bp, Artificial Sequence
  siRNA
gtgagataatttcggttcagt

SEQ ID NO: 17 - DNA, 21 bp, Artificial Sequence
  siRNA
gctttatgagatacgagtcca

SEQ ID NO: 18 - DNA, 21 bp, Artificial Sequence
  siRNA
gccaagtaataacccaagaat

SEQ ID NO: 19 - DNA, 29 bp, Artificial Sequence
  shRNA
gctcatacatcctatgagatttctgcctg

SEQ ID NO: 20 - DNA, 29 bp, Artificial Sequence
  shRNA
gtacatctctagcagtgctggagccaggt

SEQ ID NO: 21 - DNA, 23 bp, Artificial Sequence
  gRNA
cagtagcgttcgcccgaacatgg

SEQ ID NO: 22 - DNA, 23 bp, Artificial Sequence
  gRNA
gttcgggcgaacgctactgcagg

SEQ ID NO: 23 - DNA, 23 bp, Artificial Sequence
  gRNA
cgggcgaacgctactgcaggtgg

SEQ ID NO: 24 - DNA, 23 bp, Artificial Sequence
  gRNA
tgctccgtgtcgccatgttcggg

SEQ ID NO: 25 - DNA, 23 bp, Artificial Sequence
  gRNA
ttcgcccgaacatggcgacacgg

SEQ ID NO: 26 - DNA, 23 bp, Artificial Sequence
  gRNA
ctgctccgtgtcgccatgttcgg

SEQ ID NO: 27 - DNA, 23 bp, Artificial Sequence
  gRNA
cgggcggcagcagtgcgaccagg

SEQ ID NO: 28 - DNA, 23 bp, Artificial Sequence
  gRNA
ccggagctctctgcgaagtctgg

SEQ ID NO: 29 - DNA, 23 bp, Artificial Sequence
  gRNA
tctgcgaagtctggacgcagagg

SEQ ID NO: 30 - DNA, 23 bp, Artificial Sequence
  gRNA
cgaccagggtgaataggaacggg

SEQ ID NO: 31 - DNA, 23 bp, Artificial Sequence
  gRNA
aggagagaatgtgcagcgcgagg

SEQ ID NO: 32 - DNA, 23 bp, Artificial Sequence
  gRNA
actcccgttcctattcaccctgg

SEQ ID NO: 33 - DNA, 23 bp, Artificial Sequence
  gRNA
gcagtgcgaccagggtgaatagg

SEQ ID NO: 34 - DNA, 23 bp, Artificial Sequence
  gRNA
agaatgtgcagcgcgaggccagg

SEQ ID NO: 35 - DNA, 23 bp, Artificial Sequence
  gRNA
acatggcgacacggagcagcagg

SEQ ID NO: 36 - DNA, 23 bp, Artificial Sequence
  gRNA
gaccgcgcgcgccgctcccgggg

SEQ ID NO: 37 - DNA, 23 bp, Artificial Sequence
  gRNA
gggcggcagcagtgcgaccaggg

SEQ ID NO: 38 - DNA, 23 bp, Artificial Sequence
  gRNA
acttcgcagagagctccgggcgg

SEQ ID NO: 39 - DNA, 23 bp, Artificial Sequence
  gRNA
tcgcgctgcacattctctcctgg

SEQ ID NO: 40 - DNA, 23 bp, Artificial Sequence
  gRNA
ctggtcgcactgctgccgcccgg

SEQ ID NO: 41 - DNA, 23 bp, Artificial Sequence
  gRNA
gcgaccagggtgaataggaacgg

SEQ ID NO: 42 - DNA, 23 bp, Artificial Sequence
  gRNA
gggaccgcgcgcgccgctcccgg

SEQ ID NO: 43 - DNA, 23 bp, Artificial Sequence
  gRNA
cgcgcgcggtcccggcccagcgg

SEQ ID NO: 44 - DNA, 23 bp, Artificial Sequence
  gRNA
ggaccgcgcgcgccgctcccggg

SEQ ID NO: 45 - DNA, 23 bp, Artificial Sequence
  gRNA
cagacttcgcagagagctccggg

SEQ ID NO: 46 - DNA, 23 bp, Artificial Sequence
  gRNA
ccagacttcgcagagagctccgg

SEQ ID NO: 47 - DNA, 23 bp, Artificial Sequence
  gRNA
caggccgcccgcgccgctccggg

SEQ ID NO: 48 - DNA, 23 bp, Artificial Sequence
  gRNA
gggagcggcgcgcgcggtcccgg

SEQ ID NO: 49 - DNA, 23 bp, Artificial Sequence
  gRNA
tgcacattctctcctggcggcgg

SEQ ID NO: 50 - DNA, 23 bp, Artificial Sequence
  gRNA
gcgaggccaggagagccgctggg

SEQ ID NO: 51 - DNA, 23 bp, Artificial Sequence
  gRNA
cgcgaggccaggagagccgctgg

(19) United States
(12) Patent Application Publication
(10) Pub. No.: US 2019/0169620 A1
(43) Pub. Date: Jun. 6, 2019

(54) Treatment of HER2-Dependent Cancers by SORL1 Inhibition

(71) Applicant: TURUN YLIOPISTO, Turun yliopisto (FI)

(72) Inventors: Mika PIETILÄ, Naantali (FI); Johanna IVASKA, Turku (FI); Pranshu SAHGAL, Turku (FI)

(73) Assignee: TURUN YLIOPISTO, Turun yliopisto (FI)

(21) Appl. No.: 16/146,448

(22) Filed: Sep. 28, 2018

(30) Foreign Application Priority Data
Dec. 1, 2017 (FI) 20176083

(51) Int. Cl. C12N 15/113; A61K 45/06; C12N 9/22; A61K 39/395; A61P 35/00

(52) U.S. Cl. CPC C12N 15/1138; A61K 45/06; C12N 9/22; A61K 39/39558; C12N 2800/80; C12N 2310/14; C12N 2310/122; C12N 2310/20; A61P 35/00

(57) ABSTRACT

This disclosure relates to the field of cancer therapeutics. Exemplary embodiments relate to agents that inhibit expression of SORL1, a gene encoding for Sortilin related protein family-A (SORLA) or the function of SORLA protein, a multifunctional protein belonging to sortilin and LDL-receptor families, and to methods for their use in treating HER2-dependent cancers.

### FIELD OF THE INVENTION

This invention relates to the field of cancer therapeutics.

### BACKGROUND OF THE INVENTION

Human epidermal growth factor receptor family (HER) consists from epidermal growth factor receptor (EGFR), HER2 (ErbB2) HER3 (ErbB3) and HER4 (ErbB4). HER2 is a well-established oncogene especially in breast cancer, where the amplification of HER2 is found in 15-30% of patients. Moreover, HER2 overexpression or activating mutations occurs in many other solid tumors, such as lung adenocarcinoma, bladder cancer and ovarian cancer, but also breast cancers classified as HER2 negative (HER2−; corresponding to tumors with normal, not overexpressed or amplified, levels of HER2 expression may contain activating somatic mutations (about 2% of the HER2− breast cancers). The first targeted therapy against HER2, namely Trastuzumab, had tremendous impact on the survival of the patients with HER2 amplification. However, the Trastuzumab as a single agent led still to significant proportion of patients that did not respond to treatment. Development and clinical use of next-generation monoclonal antibodies against HER2 (Pertuzumab), antibody-cytotoxic drug conjugate (T-DM1) and kinase inhibitors (Lapatinib) has demonstrated the benefit of targeting HER2 with multiple different ways either with a combination of different targetting modalities during the first-line treatment or as a single-agent after the first-line treatment has failed. Identification of novel vulnerabilities of HER2 is highly important to increase the future anti-HER2 therapy repertoire.

### BRIEF DESCRIPTION OF THE INVENTION

The invention is based on the observation that SORLA is highly expressed in HER2 amplified breast cancer cell lines. In addition, sortilin-related receptor with A-type repeats (SORLA) was identified in an in vitro receptor trafficking siRNA screen as a potential regulator of receptor traffic in breast cancer cells. As SORLA has not been implicated in human cancer previously this unexpected observation was pursued. Further investigation revealed a surprising link between SORLA expression and proliferation of HER2 amplified cancers such as breast cancers. Thus, SORLA is a novel regulator of HER2 function necessary for proliferation and tumorigenesis of HER2-dependent cancers.

One aspect of the present invention is directed to a SORL1 inhibiting agent for use in treating HER2-dependent cancer, as defined in claim 1. Some other aspects of the present invention are directed to a drug combination comprising the agent of claim 1.

Further aspects, embodiments, details and advantages of the present invention will become apparent from the following figures, detailed description, examples and dependent claims.

### BRIEF DESCRIPTION OF THE DRAWINGS

In the following the invention will be described in greater detail by means of preferred embodiments with reference to the attached drawings, in which

FIGS. 1A-1F. SORLA is mainly expressed in HER2 amplified breast cancer cell lines and correlates with HER2 in bladder cancer clinical specimens. FIG. 1A) Western blot analysis of SORLA and HER2 in panel of breast cancer cell lines. FIG. 1B) Western blot analysis of SORLA and HER2 in panel of bladder cancer cell lines. FIG. 1C) Western blot analysis of SORLA and HER2 levels from normal mammary glands and tumors from the MMTV-Neu mice. FIG. 1D) Histological staining of SORLA and HER2 from bladder cancer tumor microarray (TMA; totally 199 patients) showing significant correlation of SORLA and HER2 (chi-square test p=0.0079). FIG. 1E) Histological staining of SORLA and HER2 from breast cancer TMA (totally 883 patients). FIG. 1F) In silico biomarker assessment tool (km-plot.com; including all the data sets from 2010, 2012, 2014, 2017) analysis showing Kaplan-Meier plots of overall survival (OS; 10 years) and relapse free survival (RFS; 20 years) of SORLA high and SORLA low (patients split by the best median cutoff) within all the breast cancers (RFS n=3955; OS n=1402) and within HER2 amplified breast cancers (RFS n=252; OS n=129).

FIGS. 2A-2J. SORLA regulates in vitro proliferation and in vivo tumor engraftment of HER2 dependent cancer cells irrespective of their anti-HER2 therapy response. FIG. 2A) Proliferation curves of Trastuzumab and/or Lapatinib sensitive (indicated as blue) BT474 and SKBR3 cell lines as well as Trastuzumab and/or Lapatinib resistant (indicated as red) HCC1954 and MDA-MB-361 cell lines after SORLA silencing. FIG. 2B) Rescue proliferation experiment with MDA-MB-361 SORLA-GFP and GFP-ctrl cells silenced with siRNA against the 3′UTR of SORLA. FIG. 2C) Proliferation curves of bladder cancer cell line 5637 (HER2 activating mutation S310F) and FGFR2 amplified breast cancer cell line MFM-223 after SORLA silencing. FIG. 2D) Proliferation curve of parental, GFP-ctrl and SORLA-GFP overexpressing JIMT-1 cells. In all the proliferation assays results are represented as mean±SEM and * indicates Unpaired T-test p<0.05 after 8d cell growth, ** indicates Unpaired T-test p<0.01 after 8d cell growth, *** indicates Unpaired T-test p<0.001 after 8d cell growth, **** indicates Unpaired T-test p<0.0001 after 8d cell growth and N.S. is abbreviation from non-significant. FIG. 2E) Colony formation assay of shCTRL, shSORLA #1 and shSORLA #4 BT474 and MDA-MB-361 cells. Results are represented as mean±SEM. FIG. 2F) Soft-agar colony formation assay of BT474 cells stably expressing shRNA against SORLA (two individual shRNAs; shSORLA #1 and shSORLA #4) and scramble (shCTRL). Results are represented as median±min to max. FIG. 2G) in ovo chorioallantoic membranes (CAM) tumor formation assay of BT474 cells transiently transfected with siRNAs against SORLA (siSORLA #4) and scramble (siCTRL). After 6 day of tumor growth, tumors were imaged by stereo microscope and tumor weight was measured after dissecting tumors. Results are represented as median±min to max. FIG. 2H) Subcutaneous tumor growth of transiently SORLA silenced (siSORLA #3) and scramble (siCTRL) silenced 5637 cells in nude mice. After 24 days of growth, tumors were dissected and tumor area was calculated with the following formula: (w²×w²×l²)/2. Results are represented as mean±SEM. FIG. 2I) Histological staining of KI-67 and quantification of KI-67 positive tumor areas from the subcutaneous in vivo tumor growth model of SORLA (siSORLA #3) and scramble (siCTRL) silenced 5637 cells. Results are represented as median±min to max. FIG. 2J) In vivo tumor engraftment of shSORLA and shCTRL MDA-MB-361 cells injected into mammary ducts of nod scid mice (100 000 cells per mice) mimicking the ductal carcinoma in situ (DCIS). After 10 weeks of tumor growth, number of DCIS tumors were quantified from Carnoy-staining of mammary glands. Results are represented as mean±SEM.

FIGS. 3A-3I. SORLA co-localizes and associates with HER2 on plasma membrane and in EEA1 or VPS35 positive endosomal structures via its ECD. FIG. 3A) Immunofluorescent staining of endogeneous SORLA and HER2 in MDA-MB-361. FIG. 3B) Immunofluorescent staining of endogenous EEA-1, SORLA and HER2 in MDA-MB-361 as well as endogenous staining of HER2 and VPS35 in SORLA-GFP JIMT-1 cells. FIG. 3C) Live cell TIRF imaging of Trastuzumab conjugated with AlexaFluor-568 (Tz-568) and SORLA-GFP in MDA-MB-361 cells. FIG. 3D) Live cell intracellular imaging of Trastuzumab conjugated with AlexaFluor-568 (Tz-568) and SORLA-GFP in MDA-MB-361 cells. FIG. 3E) Co-immunoprecipitation of endogenous HER2 in SORLA-GFP and GFP-ctrl SKBR3 cells after GFP-pulldown. FIG. 3F) Co-immunoprecipitation of endogenous SORLA after endogenous HER2 pulldown in BT474 and MDA-MB-361 cells FIG. 3G) Schematic overview of the SORLA protein structures and domains. FIG. 3H) Confocal imaging of different SORLA fragments to study the localization. SORLA-GFP TM+CD indicates SORLA-GFP truncated form lacking the entire extracellular domain; SORLA-GFP ECD+TM indicates SORLA-GFP truncated form lacking the cytosolic domain. FIG. 3I) Rescue proliferation assay of GFP-control, TM+CD, ECD+TM and full length overexpressing MDA-MB-361 cells silenced with scramble and SORLA 3′UTR targeting siRNA. Results are shown as mean±SEM of four technical replicates from one independent experiment. Unpaired student-test was used as statistical test.

FIGS. 4A-4K. SORLA promotes plasma membrane retention of HER2. FIG. 4A) Representative western blots of HER2 protein levels after SORLA silencing or SORLA-GFP overexpression. FIG. 4B) Quantification of HER2 protein levels from three independent replicates (mean±SEM). FIG. 4C) Flow cytometry analysis of cell surface HER2 levels from three independent replicates (mean±SEM) after SORLA silencing or SORLA-GFP overexpression. FIG. 4D) Representative confocal images of HER2 localization after SORLA silencing in BT474 cells. FIG. 4E) Quantification of intracellular HER2 signal from SORLA silenced BT474 cells (median±min to max). FIG. 4F) Trastuzumab internalization assay (Trastuzumab conjugated with AlexaFluor 568; Tz-568) in MDA-MB-361 SORLA silenced and scramble silenced cells. Representative images of SORLA silenced and control silenced cells after cell surface labeling with Tz-568 and following internalization after 0 min, 15 min, 30 min and 60 min of incubation. G) Quantification of intracellular Tz-568 signal in different time points in MDA-MB-361 SORLA silenced and control silenced cells (median±min to max). FIG. 4H) Representative blots of biotin-based HER2 internalization assays in MDA-MB-361 cells after SORLA silencing and JIMT-1 after overexpression of SORLA-GFP. FIG. 4I) Quantification of HER2 internalization from two independent experiment for MDA-MB-361 (mean±SEM) and five independent experiment for JIMT-1 (mean±SEM). FIG. 4J) Immunofluorescent imaging of LAMP1 and HER2 in MDA-MB-361 shCTRL, shSORLA #1 and shSORLA #4 cells. FIG. 4K) Western blot analysis of HER2 down-stream signaling after SORLA silencing in MDA-MB-361 and BT474 cells.

FIG. 5A-5J. Impaired lysosomal function of HER2 amplified breast cancer cells after SORLA silencing. FIG. 5A) Immunofluorescent imaging of LAMP1 and CD63 (LAMP3) in BT474 and MDA-MB-361 cells after SORLA silencing. FIG. 5B) Quantification of lysosomal aggregation after SORLA silencing in MDA-MB-361 and BT474 cells. Data is represented as median±min to max. FIG. 5C) Transmission electron microscope (TEM) imaging of lysosomes after SORLA silencing in MDA-MB-361 and BT474 cells. FIG. 5D) Representative images of DQ-BSA stained MDA-MB-361 cells after SORLA silencing. FIG. 5E) Flow cytometry analysis of DQ-BSA signal after SORLA silencing in MDA-MB-361 cell (mean±SD). FIG. 5F) Subcellular fractionation into cytosolic and nuclear fractions in BT474 after SORLA silencing to study the localization of TFEB. FIG. 5G) Quantitative real time PCR analysis of TFEB target genes after SORLA silencing in BT474 cells (mean±SEM). FIG. 5H) Representative images of wild type TFEB localization in MDA-MB-361 after starvation (siCTRL cells) and after SORLA silencing. Transfection of TFEB-S3A,R4A double mutant was used as a positive control to study nuclear localization of TFEB. FIG. 5I) Quantification of nuclear/cytoplasmic signal ratio of TFEB (median±min to max). FIG. 5J) Quantitative real time PCR analysis of TFEB target genes after SORLA silencing in MDA-MB-361 cells (mean±SEM).

FIGS. 6A-6C. Combination of Ebastine with SORLA silencing gives synergistic effect on cell viability of HER2 dependent cancer cells. FIG. 6A) Colony formation assay of SORLA silenced and control silenced 5637 cells treated with different concentrations of Ebastine and Loratadine for 7 days. FIG. 6B) Quantification of confluency from the colony formation assays by using ImageJ Colony Area Plug-in. Results are shown as mean±SD. * indicates Unpaired T-test p<0.05, ** Unpaired T-test p<0.01, *** indicates Unpaired T-test p<0.001, **** indicates Unpaired T-test p<0.0001. FIG. 6C) Cell viability assay of SORLA silenced cells treated with a range of different concentrations of Ebastine for 48 hours. IC50 values were counted from three independent experiments (mean±SEM).

FIG. 7A-7D. Targeting HER2 amplified breast cancer cells with SORLA targeting monoclonal antibody. FIG. 7A) Relative viability of IgG or SORLA targeting antibody treated BT474, HCC1954 and MDA-MB-361 cells. Results are shown as mean±SEM of four technical replications and unpaired student-test was used as statistical test. FIG. 7B) Relative viability of IgG or SORLA targeting antibody treated MDA-MB-231 and MCF10A cells. Results are shown as mean±SEM of four technical replications and unpaired student-test was used as statistical test. FIG. 7C) SORLA-binding antibody sensitizes anti-HER2 therapy (Trastuzumab) resistant cells to therapy in vitro. Relative viability of IgG, SORLA targeting antibody or Trastuzumab treated MDA-MB-361 cells. The combination of SORLA targeting antibody with trastuzumab shows additive effect on cell viability of MDA-MB-361 cells. Results are shown as mean±SD of four biological replications and unpaired student-test was used as statistical test. FIG. 7D) SORLA-binding antibody sensitizes anti-HER2 therapy (Trastuzumab) resistant cells to therapy in vivo. in ovo chorioallantoic membranes (CAM) tumor formation assay of MDA-MB-361 cells treated IgG, SORLA targeting antibody or Trastuzumab (treatments at day 0 and day 3; 9 μg/1×106 cells (450 μg/ml) or 2.25 μg/1×106 cell (112.5 μg/ml, ¼). After 5 day of tumor growth, tumor weight was measured after dissecting tumors. The combination of SORLA targeting antibody with trastuzumab shows additive effect on tumor growth. N=18, 18, 20, 16 and 10. Results are represented as median±min to max.

FIG. 8A) Scoring of SORLA and EGFR protein expression in bladder cancer TMA. FIG. 8B) TCGA analysis of correlation between SORLA and HER2 in different cancer types.

FIGS. 9A-9K. FIG. 9A) Western blot analysis of SORLA protein levels after SORLA silencing with five individual siRNAs targeting different parts of SORLA mRNA in BT474 cells. FIG. 9B) Proliferation assay of SORLA silenced BT474 cells when compared to siCTRL. Results are represented as a mean±SD of four replicates. FIG. 9C) Western blot analysis of SORLA protein levels after silencing SORLA with two individual siRNAs in BT474, SKBR3, HCC1954, MDA-MB-361, 5637 and MFM-223 cells. FIG. 9D) Western blot analysis of SORLA protein levels after silencing SORLA with siRNA targeting 3′UTR of SORLA mRNA in MDA-MB-361 GFP-ctrl and MDA-MB-361 SORLA-GFP cells. FIG. 9E) Western blot analysis of SORLA in JIMT-1 GFP-ctrl and JIMT-1 SORLA-GFP cells. FIG. 9F) Proliferation assay of BT474 during lapatinib treatment. FIG. 9G) Cell cycle analysis of SORLA silenced and Lapatinib treated (0.5 μM for 24 h) BT474 cells. FIG. 9H) Efficient SORLA silencing with two individual shRNA in BT474 and MDA-MB-361 cells. FIG. 9I) Proliferation assay of MDA-MB-361 and BT474 cells expressing shRNA against SORLA (two individual shRNA: shSORLA #1 and shSORLA #4) or scramble shRNA (shCTRL). FIG. 9J) Western blot analysis of SORLA protein levels to confirm the efficiency of SORLA silencing in BT474 used for CAM assay. FIG. 9K) Western blot analysis of SORLA protein levels to confirm the efficiency of SORLA silencing in 5637 cells used for subcutaneous in vivo tumor growth model.

FIG. 10A-10C. FIG. 10A) Co-localization studies of SORLA-GFP and endogenous SORLA with different markers of endosomal compartments. FIG. 10B) Immunofluorescent staining of endogenous HER2 in panel of HER2 amplified breast cancer cell lines together with early endosome marker EEA-1. FIG. 10C) Immunofluorescent staining of endogenous HER2 in BT474 after Primaquine or vehicle treatment (24 h, 0.3 and 0.6 mM).

FIG. 11. Quantitative real time PCR analysis of ErbB2 gene expression levels after stable SORLA silencing in BT474 with two individual shRNAs (shSORLA #1 and shSORLA #4) or scramble (shCTRL) as well as in JIMT-1 GFP-Ctrl and SORLA-GFP cells. Results are represented as mean±SD of three independent replicates.

FIGS. 12A-12F. FIG. 12A) Immunofluorescent staining of LAMP1 after SORLA silencing with four individual siRNAs (siSORLA #1, siSORLA #2, siSORLA #3 and siSORLA #4) or with scramble (siCTRL) in BT474 and MDA-MB-361 cells. FIG. 12B) Immunofluorescent staining of LAMP1 after Lapatinib treatment (24 h, 0.5 mM) or HER2 silencing with two individual siRNAs (siHER2 #2, siHER2 #4) or with scramble in BT474 and MDA-MB-361 cells. FIG. 12C) Quantification of (perinuclear) aggregation of LAMP1 positive late endosomes/lysosomes after Lapatinib treatment or HER2 silencing in BT474 and MDA-MB-361 cells. Results are represented as median±min to max. FIG. 12D) Proliferation assay of HER2 silenced and lapatinib treated MDA-MB-361 cells. Results are represented as mean±SD of four replicates. FIG. 12E) Immunofluorescent staining of LAMP1 after SORLA silencing in HER2 negative MFM-223 cells. FIG. 12F) Western blot analysis of TFEB and LAMP1 protein levels after SORLA (siSORLA #1, siSORLA #2, siSORLA #3 and siSORLA #4) silencing and scramble silencing in MDA-MB-361 cells or after SORLA silencing with two individual siR-NAs against SORLA (siSORLA #3 and siSORLA #4) or scramble in SKBR3 and BT474 cells. Western blot analysis of LAMP1 protein levels after Lapatinib treatment (24 h).

FIG. 13A) Representative crystal violet staining after 7 days Chloroquine and Penfluridol treated SORLA silenced (siSORLA #3 and siSORLA #4) or scramble silenced (siCTRL) 5637 cells. FIG. 13B) Toxicity assay of BT474 and MDA-MB-361 SORLA silenced (siSORLA #3 and siSORLA #4) or scramble silenced (siCTRL) treated with increased amount of Loratadine. Results are represented as mean±SD of four replicates.

### DETAILED DESCRIPTION OF THE INVENTION

The present invention relates to agents that inhibit expression of SORL1, a gene encoding for Sortilin related protein family-A (SORLA) or the function of SORLA protein, a multifunctional protein belonging to sortilin and LDL-receptor families, and to their use in treating HER2-dependent cancers.

Accordingly, the invention also relates to a method of treating HER2-dependent cancer in a subject in need thereof by administering at least one SORL1 inhibiting agent to said subject.

As used herein, the term “SORL1 inhibiting agent” refers to any agent that silences or down-regulates the expression of SORL1 gene, edits SORL1 by targeted gene disruption, or blocks or interferes with the function of SORLA.

As used herein, the term “SORL1 silencing” refers to complete or partial reduction of SORL1 gene expression. In some embodiments, SORL1 gene expression is reduced e.g. by at least 50%, or at least 80%, 81%, 82%, 83%, 84%, 85%, 86%, 87%, 88%, 89%, 90%, 91%, 92%, 93%, 94%, 95%, 96%, 97%, 98%, 99%, or 100% when a SORL1 silencing agent is introduced into a human or animal subject.

SORL1 silencing may be obtained by any suitable method or means known in the art including, but not limited to, RNA interference (RNAi), CRISPR/CAS9 or other form of gene editing and ribozymes that cleave the SORL1 mRNA. The ribozyme technology is described, for example, by Li et al. in Adv. Cancer Res., 2007, 96:103-43.

The most common approach for RNAi-based gene silencing is the use of small interfering RNA (siRNA). The principle of siRNA is extensively presented in literature. As examples can be mentioned the US patent publications 2003/0143732, 2003/0148507, 2003/0175950, 2003/0190635, 2004/0019001, 2005/0008617 and 2005/0043266. An siRNA duplex molecule comprises an antisense region and a sense strand wherein said antisense strand comprises nucleotide sequence complementary to a target region in an mRNA sequence encoding a certain protein, and the sense strand comprises nucleotide sequence complementary to the said antisense strand. In other words, siRNAs are small double-stranded RNAs (dsRNAs). The sense strand and antisense strand can be covalently connected via a linker molecule, which can be a polynucleotide linker or a non-nucleotide linker. The length of the antisense and sense strands may vary and is typically about 19 to 21 nucleotides each. In some cases, the siRNA may comprise 22, 23 or 24 nucleotides.

Another approach for RNAi-based SORL1 silencing is to use longer, typically 25-35 nt, Dicer substrate siRNAs (DsiRNAs), which in some cases have been reported to be more potent than corresponding conventional 21-mer siR-NAs (Kim et al., Nat Biotechol, 2005, 23: 222-226). DsiRNAs are processed in vivo into active siRNAs by Dicer. In a cell, an active siRNA antisense strand is formed and it recognizes a target region of the target mRNA. This in turn leads to cleaving of the target RNA by the RISC endonuclease complex (RISC=RNA-induced silencing complex) and also in the synthesis of additional RNA by RNA dependent RNA polymerase (RdRP), which can activate Dicer and result in generation of additional siRNA duplex molecules, thereby amplifying the response.

As used herein, the term “small double-stranded RNA” (dsRNA) refers to both siRNAs and DsiRNAs.

Typically, but not necessarily, the antisense strand and the sense strand of dsRNA both comprise a 3′-terminal overhang of a few, typically 1 to 3 nucleotides. The 3′ overhang may include one or more modified nucleotides, such as a 2′-O-methyl ribonucleotide. The 5′-terminal of the antisense is typically a phosphate group (P). The dsRNA duplexes having terminal phosphate groups (P) are easier to administrate into the cell than a single stranded antisense. In some cases, the 5′-terminal of the sense strand or of both antisense and sense strands may comprise a P group.

Artificial microRNA (miRNA) precursors are another class of small RNAs suitable for mediating RNAi. Typically, artificial miRNA precursors are about 21-25 nucleotides in length, and they may have 1 to 3, typically 2, overhanging 3′ nucleotides.

Short-hairpin RNAs (shRNAs) are still another way of silencing SORL1 by RNAi. shRNAs consist of i) a short nucleotide sequence, typically ranging from 19 to 29 nucleotides, derived from the target gene; ii) a loop, typically ranging between 4 to 23 nucleotides; and iii) a short nucleotide sequence reversely complementary to the initial target sequence, typically ranging from 19 to 29 nucleotides.

SORL1 silencing may also be obtained by antisense therapy, where relatively short (typically 13-25 nucleotides) synthetic single-stranded DNA or RNA oligonucleotides inactivate SORL1 gene by binding to a corresponding mRNA. Antisense oligonucleotides may be unmodified or chemically modified. In some embodiments, the hydrogen at the 2′-position of ribose is replaced by an O-alkyl group, such as methyl. In further embodiments, antisense oligonucleotides may contain one or more synthetic or natural nucleotide analogs including, but not limited to peptide-nucleic acids (PNAs).

Delivery of SORL1 specific RNAi molecules can be accomplished in two principally different ways: 1) endogenous transcription of a nucleic acid sequence encoding the oligonucleotide, where the nucleic acid sequence is located in an expression construct or 2) exogenous delivery of the oligonucleotide.

For endogenous transcription, SORL1 specific RNAi molecules may be inserted into suitable expression systems using methods known in the art. Non-limiting examples of such expression systems include retroviral vectors, adenoviral vectors, lentiviral vectors, other viral vectors, expression cassettes, and plasmids, such as those encapsulated in pegylated immunoliposomes (PILs), with or without one or more inducible promoters known in the art. If dsRNA is employed, both RNA strands may be expressed in a single expression construct from the same or separate promoters, or the strands may be expressed in separate expression constructs.

Typically, expression constructs are formulated into pharmaceutical compositions prior to administration to a human or animal subject. Administration may be performed by any suitable method known in the art, including systemic and local delivery. The formulation depends on the intended route of administration as known to a person skilled in the art. By way of example, the expression construct may be delivered in a pharmaceutically acceptable carrier or diluent, or it may be embedded in a suitable slow release composition. In some cases, the pharmaceutical composition may contain one or more cells producing the expression construct. Also bacteria may be used for RNAi delivery. For instance, recombinantly engineered Escherichia coli can enter mammalian cells after in vivo delivery and transfer shRNAs. A related approach is to use minicells derived e.g. from Salmonella enterica.

For exogenous delivery, RNAi molecules are typically complexed with liposome or lipid-based carriers, cholesterol conjugates, or polyethyleneimine (PEI). A promising new approach is to complex dsRNAs with stable nucleic acid lipid particles (SNALPs). Suitable routes of administration for exogenous delivery, with or without said complexing, include, but are not limited to, parenteral delivery (e.g. intravenous injection), enteral delivery (e.g. orally), local administration, topical administration (.e.g. dermally or transdermally) as known to a person skilled in the art. Since surgical removal of a tumour is usually the primary clinical intervention, RNAi molecules may be administered directly to the resected tumour cavity.

Normal, unmodified RNA has low stability under physiological conditions because of its degradation by ribonuclease enzymes present in the living cell or biological fluid. If the oligonucleotide shall be administered exogenously, it is highly desirable to modify the molecule according to known methods so as to enhance its stability against chemical and enzymatic degradation.

Modifications of nucleotides to be administered exogenously in vivo are extensively described in the art (e.g. in US 2005/0255487, incorporated herein by reference). Principally, any part of the nucleotide, i.e. the ribose sugar, the base and/or internucleotidic phosphodiester strands can be modified. For example, removal of the 2′-OH group from the ribose unit to give 2′-deoxyribosenucleotides results in improved stability. Prior disclosed are also other modifications at this group: the replacement of the ribose 2′-OH group with alkyl, alkenyl, allyl, alkoxyalkyl, halo, amino, azido or sulfhydryl groups. Also other modifications at the ribose unit can be performed: locked nucleic acids (LNA) containing methylene linkages between the 2′- and 4′-positions of the ribose can be employed to create higher intrinsic stability.

Furthermore, the internucleotidic phosphodiester linkage can, for example, be modified so that one or more oxygen is replaced by sulfur, amino, alkyl or alkoxy groups. Also the base in the nucleotides can be modified.

Preferably, the oligonucleotide comprises modifications of one or more 2′-hydroxyl groups at ribose sugars, and/or modifications in one or more internucleotidic phosphodiester linkages, and/or one or more locked nucleic acid (LNA) modification between the 2′- and 4′-position of the ribose sugars.

Particularly preferable modifications are, for example, replacement of one or more of the 2′-OH groups by 2′-deoxy, 2′-O-methyl, 2′-halo, e.g. fluoro or 2′-methoxyethyl. Especially preferred are oligonucleotides where some of the inter-nucleotide phoshodiester linkages also are modified, e.g. replaced by phosphorothioate linkages.

In some embodiments, RNAi molecules may contain one or more synthetic or natural nucleotide analogs including, but not limited to, phosphorothioates, phosphoramidates, methyl phosphonates, chiral-methyl phosphonates, and peptide-nucleic acids (PNAs) as long as dsRNAs retain their SORL1 silencing ability.

It should be stressed that the modifications mentioned above are only non-limiting examples.

One of the challenges related to RNAi is the identification of a potent RNAi molecule for the corresponding mRNA. It should be noted that genes with incomplete complementarity are inadvertently downregulated by the RNAi, leading to problems in data interpretation and potential toxicity. This however can be partly addressed by carefully designing appropriate RNAi molecules with design algorithms. These computer programs sieve out given target sequence with a set of rules to find sequence stretches with low GC content, a lack of internal repeats, an A/U rich 5-end and high local free binding energy which are features that enhance the silencing effect of dsRNA.

In order to identify agents useful in the present invention, SORL1 silencing RNAi molecules can be designed by using commercial or non-commercial algorithms available in the art. This may be achieved e.g. by loading the full length cDNA sequence of SORL1 to an algorithm program. Algorithm-generated RNAi sequences can then screened trough genome wide DNA sequence alignment (BLAST) to eliminate RNAi molecules which are not free from off-targeting. In other words, all those RNAi molecules which have even short sequence regions matching with other genes than target gene (SORL1) may be considered invaluable for further use. Non-limiting examples of algorithm programs suitable for designing siRNAs include Eurofins MWG Operon's Online Design Tool or a stand-alone program developed by Cuia et al. (Biomedicine, 2004, 75: 67-73). Algorithm programs suitable for designing other types of RNAi molecules, such as shRNA and miRNA molecules, are also readily available in the art.

Obtained RNAi molecules can then be synthetized and transfected to different cell lines and their capacity to degrade mRNA and further deplete translation of SORL1 can be studied at protein level by measuring the amount of SORLA protein after siRNA treatment with SORLA specific antibodies or by analysing mRNA levels of SORL1 with sequencing or q-RT-PCR.

Non-limiting examples of dsRNA sequences suitable for silencing SORL1 include those that comprise a sequence selected from the group consisting of SEQ ID Nos: 1-18 while non-limiting examples of shRNA sequences suitable for silencing SORL1 include those that comprise a sequence selected from the group consisting of SEQ ID Nos: 19 and 20. Still further SORL1 specific RNAi sequences suitable for use in various embodiments of the present invention can be designed and synthetized according to methods known in the art. Any such isolated RNAi sequence must be sufficiently complementary to SORL1 mRNA sequence in order to silence SORL1 gene but lack significant off-targeting. This means that although 100% complementarity is preferred, also RNAi sequences with lower complementarity may be suitable for use in the present invention. Those skilled in the art are able to determine the required complementarity for each case.

The term “complementary” is well known in the art and it means Watson-Crick base pairing where nucleobase adenine (A) in a target motif sequence is represented by nucleobase thymine (T) in a corresponding binding unit, or vice versa. Accordingly, nucleobase cytosine (C) in a target motif is represented by nucleobase guanine (G) in a corresponding binding unit, or vice versa. In other words, the complementary sequence to, for instance, 5′-T-T-C-A-G-3′ is 3′-A-A-G-T-C-5′. As is readily understood by those skilled in the art, RNA differs from DNA by containing uracil (U) instead of T. Uracil is complementary to adenine.

Accordingly, although the most preferred siRNA and shRNA sequences may, at least in some embodiments, be those having 100% sequence identity with SEQ ID NOs: 1-20, also siRNAs and shRNA having lower sequence identity are envisaged. Accordingly, suitable siRNA and shRNA sequences include also those having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity with SEQ ID NO:s 1-20, as long as they have similar binding properties and SORL1 silencing activity as the reference RNAi molecules. One aspect of the invention relates to such siRNA and shRNA molecules.

As used herein, the percent identity between two nucleic acid sequences is equivalent to the percent homology between the two sequences. The percent identity between the two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using standard methods known in the art.

In some embodiments, SORL1 inhibition may be contemplated by a nuclease system comprising at last one genome targeted nuclease and at least one guide RNA comprising at least one targeted genomic sequence. Preferably, the nuclease system is Clustered Regularly Interspaced Short Palindromic Repeats (CRISPR) and CRISPR-associated endonuclease protein (cas) system, i.e. CRISPR-Cas system, preferably CRISPR-Cas9 system.

As used herein, the term “guide RNA” (gRNA) molecule refers to a short synthetic nucleic acid molecule that promotes the specific targeting or homing of a gRNA molecule/Cas molecule complex to a target nucleic acid. In other words, gRNA provides both targeting specificity and scaffolding/binding ability for Cas9 nuclease. To this end, gRNA is composed of a “scaffold” sequence necessary for Cas9-binding and a user-defined ˜20 nucleotide “targeting domain” which defines the genomic target to be modified. gRNA does not exist in nature.

In certain embodiments, the gRNA molecule may be a unimolecular or chimeric gRNA consisting of a single RNA molecule. In other embodiments, the gRNA molecule may be a modular gRNA comprising more than one, and typically two, separate RNA molecules.

The present gRNA molecules comprise a targeting domain that is complementary to a target sequence in the genomic DNA encoding human SORL1. The targeting domain comprises a nucleotide sequence that is e.g., at least 80%, 85%, 86%, 87%, 88%, 89%, 90%, 91%, 92%, 93%, 94%, 95%, 96%, 97%, 98%, 99%, or 100% complementary to the target sequence on the target SORL1 nucleic acid. In some embodiments, the targeting domain may be 5 to 50, 10 to 40, 10 to 30, 15 to 30, or 15 to 25 nucleotides in length. In some more specific embodiments, the targeting domain is 15, 16, 17, 18, 19, 20, 21, 22, 23, 24 or 25 nucleotides in length. Some or all of the nucleotides of the domain can have a modification.

In some embodiments, the targeting domain is configured to provide SORL1 knockdown by introducing a frameshift mutation or a stop codon into the human genomic SORL1 DNA.

Non-limiting examples of gRNA targeting domain sequences suitable for knocking down SORL1 gene include nucleic acid sequences set forth in SEQ ID Nos: 21-51. The exemplified gRNA molecules induce potentially insertions or deletions in an area that encodes the very N-terminal part of SORLA protein, and lead to a frameshift resulting in impaired expression of SORL1. One aspect of the invention relates to gRNA molecules comprising a sequence selected from the group consisting of SEQ ID Nos: 21-51.

Further gRNA targeting domain sequences suitable for use in the present invention can be designed and analysed using software tools available in the art (e.g. the one available at http://crispr.mit.edu/). Such tools can be used to optimize the selection of gRNA within the target sequence, e.g., to minimize or predict total off-target activity across the genome. In other words, each possible gRNA can be ranked according to its total predicted off-target cleavage; the top-ranked gRNAs represent those that are likely to have the greatest on-target and the least off-target cleavage. Candidate gRNA molecules can then be validated in vitro and/or in vivo according to methods available in the art.

As used herein, the term “Cas” refers to a protein that can interact with a gRNA molecule and, in concert with the gRNA molecule, target or home to a site which comprises a target domain and a protospacer adjacent motif (PAM) sequence.

In some embodiments, the Cas protein is a Cas9 protein. As is well known in the art, Cas9 may be derived from or based on Cas9 proteins of a variety of species including, but not limited to, Streptococcus pyogenes, Streptococcus thermophilus, Staphylococcus aureus, and Neisseria meningitides. Modified Cas9 proteins with desired properties can be obtained by using any suitable means and methods available in the art.

As used herein, the term “protospacer adjacent motif” (PAM) is a sequence in the target nucleic acid. The Cas9 molecule interacts with the PAM sequence and cleaves the target nucleic acid upstream from the PAM sequence. Cas9 molecules from different bacterial species can recognize different PAM sequence motifs. For example, Streptococcus pyogenes Cas9 recognizes the sequence motif NGG, Streptococcus thermophiles Cas9 recognizes the sequence motif NGGNG and NNAGAAW (W=A or T), Staphylococcus aureus Cas9 recognizes the sequence motif NNGRR (R=A or G), whereas Neisseria meningitides Cas9 recognizes the sequence motif NNNNGATT. Cas9 directs cleavage of the target nucleic acid sequence about 20 base pairs upstream from the PAM. The ability of a Cas9 molecule to recognize a PAM sequence can be determined, e.g., using a transformation assay described in Jinek et al., Science 2012, 337:816.

Naturally occurring Cas9 molecules can recognize specific PAM sequences as explained above. Thus, in some embodiments Cas9 molecules having the same PAM specificities as naturally occurring Cas9 molecules are employed. In other embodiments, Cas9 molecules having altered PAM specificities may be employed, for example to decrease the number of off target sites and/or to improve specificity. Those skilled in the art know how to obtain such non-natural Cas molecules.

As used herein, the term “donor template” or “template nucleic acid” refers to a nucleic acid sequence which can be used in conjunction with a Cas9 molecule and a gRNA molecule to alter the structure of a target position by participating in a homology-directed repair (HDR) event. In some embodiments, the target nucleic acid is modified to have some or all of the sequence of the template nucleic acid, typically at or near cleavage site(s). For use in the present invention, a preferred template nucleic acid provides a stop codon into the target site. In some embodiments, the template nucleic acid results in the incorporation of a modified or non-naturally occurring base into the target nucleic acid.

Cas9 nucleases to be employed in the present invention may differ in their DNA cleaving properties. In some embodiments, naturally occurring Cas9 molecules having a double stranded nuclease activity, i.e., the ability to cleave both strands of a double stranded nucleic acid and create a double stranded break, are employed. Double-stranded breaks activate the doublestrand break (DSB) repair machinery. DSBs can be repaired by the cellular Non-Homologous End Joining (NHEJ) pathway, resulting in insertions and/or deletions (indels) which disrupt the targeted locus. Alternatively, if a donor template with homology to the targeted locus is supplied, the DSB may be repaired by the homology-directed repair (HDR) pathway allowing for precise replacement mutations, such as ones creating a stop codon, to be made. Such embodiments require only a single gRNA.

In some other embodiments, mutant Cas9 molecules, such as Cas9D10A or Cas9H840A, having only nickase activity may be employed. Such Cas molecules cleave only one DNA strand resulting in a single nick that does not activate NHEJ. Instead, when provided with a homologous donor template, DNA repairs are conducted via the high-fidelity HDR pathway only, increasing the ratio of HDR to NHEJ at a given cleavage site. Thus, such embodiments are more suitable for creating stop codons through donor template instead of resulting in indels.

In some further embodiments, two mutated Cas9 molecules, such as those comprising either D10A or H840A mutation, having only nickase activity may be employed together with two gRNAs, one for placement of each single strand break. Such paired Cas9 complexes do not activate NHEJ but when provided with a homologous donor template, result in DNA repairs by HDR pathway only, resulting in reduced indel mutations. Thus, such embodiments are more suitable for creating stop codons through donor template instead of resulting in indels.

In some even further embodiments, a nuclease-deficient Cas9, such as Cas9 molecule comprising both H840A and D10A mutations, may be employed. Such Cas9 molecules do not have cleavage activity, but do have DNA binding activity. Therefore, such variants can be used to sequence-specifically target any region of the genome without cleavage. Instead, by fusing with various effector domains, nuclease-deficient Cas9 can be used as a gene silencing tool by means and methods known in the art.

While the above-mentioned embodiments involve either a single double-strand break or two single strand breaks, further embodiments may involve two double stranded breaks with a break occurring on each side of the target sequence, one double stranded breaks and two single strand breaks with the double strand break and two single strand breaks occurring on each side of the target sequence, or four single stranded breaks with a pair of single stranded breaks occurring on each side of the target sequence.

The ability of a Cas9 molecule/gRNA molecule complex to bind to and cleave a target nucleic acid can be evaluated by techniques available in the art including, but not limited to, a plasmid cleavage assay and an oligonucleotide DNA cleavage assay.

In some embodiments, the nuclease, preferably Cas9, can be provided as a protein, RNA, DNA, or an expression vector comprising a nucleic acid that encodes the nuclease. In some further, embodiments, the guide RNA can be provided as an RNA molecule (gRNA), DNA molecule, or as an expression vector comprising a nucleic acid that encodes the gRNA. In some even further embodiments, the gRNA may be provided as one or more, e.g. as two, three, four, five, six, seven, eight, nine, or ten, RNA molecules (gRNA), DNA molecules, or expression vectors comprising a nucleic acid that encodes the gRNA, or any combination thereof.

Cas9-encoding and/or gRNA-encoding DNA can be administered to subjects or delivered into cells by methods well known in the art. For example, they can be delivered, e.g., by one or more vectors (e.g., viral or non-viral vectors/viruses or plasmids), non-vector based methods (e.g., using naked DNA or DNA complexes), or a combination thereof.

In accordance with the above, some embodiments of the invention relate to a vector system comprising one or more vectors, preferably one or more packaged vectors, comprising:

(a) a first regulatory or control element operably linked to a sequence encoding a gRNA as disclosed herein, and

(b) a second regulatory or control element operably linked to a nucleic acid encoding a Cas protein.

Suitable regulatory or control elements are well known in the art and include enhancers and promoters, such as regulated promoters (e.g., inducible promoters), constitutive promoters, and tissue specific promoters. The promoter can be a viral promoter or a non-viral promoter.

In some embodiments, a vector can also comprise a sequence encoding a signal peptide for targeted localization, fused to a sequence encoding the Cas9 molecule and/or the gRNA molecule. For example, a vector can comprise a nuclear localization sequence (e.g., from SV40) fused to the Cas9-encoding and/or the gRNA-encoding nucleic acid sequence.

Suitable viral vectors/viruses for use in the present invention include, but are not limited to, retroviruses, lentiviruses, adenovirus, adeno-associated virus (AAV), vaccinia viruses, poxviruses, and herpes simplex viruses.

Usually, viral vectors used in gene therapy are generated by a producer cell line that packages a nucleic acid vector into a viral particle. In some embodiments, the packaging cell line contains a helper plasmid encoding necessary viral genes. Those skilled in the art can easily select a suitable packaging cell line depending on the type of the viral vector to be used. Packaging cell lines as well as viral vectors are readily available in the art.

As set forth above, Cas9- and/or gRNA-encoding DNA may in some embodiments be delivered by a non-vector based method (e.g., using naked DNA or DNA complexes). For example, the DNA can be delivered by electroporation, gene gun, sonoporation, magnetofection, calcium phosphates, lipid-mediated transfection, or a combination thereof.

In some embodiments, the delivery vehicle may be a biological non-viral delivery vehicle such as an attenuated bacterium, a genetically modified bacteriophage, or a mammalian virus-like particle as is well known in the art.

In some other embodiments, the non-viral delivery vehicle may be a dendrimer or a nanoparticle. The nanoparticle may be an inorganic nanoparticle such as a magnetic nanoparticle (e.g., Fe₃MnO₂), or silica. The outer surface of the nanoparticle may be conjugated with a positively charged polymer (e.g., polyethylenimine, polylysine, polyserine) which allows for attachment (e.g., conjugation or entrapment) of payload. In some embodiments, the non-viral vector is an organic nanoparticle, e.g. a one that entraps the payload inside the nanoparticle. Exemplary organic nanoparticles include SNALP liposomes that contain cationic lipids together with neutral helper lipids which are coated with polyethylene glycol (PEG) and protamine and nucleic acid complex coated with lipid coating.

In some embodiments, the vehicle may have targeting modifications to increase target cell update of nanoparticles and liposomes, including but not limited to cell specific antigens, monoclonal antibodies, single chain antibodies, aptamers, polymers, sugars, and cell penetrating peptides. In some embodiments, the vehicle may use fusogenic and endosome-destabilizing peptides/polymers; while in some other embodiments, the vehicle may undergo acid-triggered conformational changes (e.g., to accelerate endosomal escape of the cargo). In some embodiments, a stimuli-cleavable polymer may be used, e.g., for release in a cellular compartment. For example, disulfide-based cationic polymers that are cleaved in the reducing cellular environment may be used.

In a preferred embodiment, the delivery vehicle may be a nanoparticle coated with an anti-HER2 antibody, such as Trastuzumab, for targeted delivery of the cargo into HER2-dependent cancer cells.

In some embodiments, SORL1 inhibiting agent is an anti-SORLA antibody. As used herein, the term “antibody” refers to an immunoglobulin structure comprising two heavy (H) chains and two light (L) chains inter-connected by disulfide bonds. Antibodies can exist as intact immunoglobulins or as any of a number of well-characterized antigen-binding fragments or single chain variants thereof, all of which are herein encompassed by the term “antibody”. Non-limiting examples of said antigen-binding fragments include Fab fragments, Fab′ fragments, F(ab′)2 fragments, Fv fragments, scFv fragments (i.e. single-chain variable fragments), nanobodies (i.e. monomeric variable domains of camelid heavy chain antibodies) and these fragments engineered to form fusions with FC region. Said fragments and variants may be produced by recombinant DNA techniques, or by enzymatic or chemical separation of immunoglobulins as is well known in the art. The term “antibody” also includes, but is not limited to, polyclonal, monoclonal, and recombinant antibodies of isotype classes IgA, IgD, IgE, IgG, and IgM and sub-types thereof. The term “antibody” also includes bispecific or dual-specificity antibodies, i.e. artificial protein that can simultaneously bind to two different types of antigen. Means and methods for producing bispecific antibodies are readily available in the art.

Preferably, the present antibodies are human or humanized antibodies. Humanized antibodies are antibodies wherein the variable region may be murine derived but which has been mutated so as to more resemble a human antibody and may contain a constant region of human origin. Fully human antibodies are antibodies wherein both the variable region and the constant region are of human origin. Means and methods for producing human and humanized antibodies are readily available in the art.

In some preferred embodiments, the anti-SORLA antibody binds specifically to the extracellular domain of SORLA and disrupts the association between SORLA and HER2. In some further embodiments said antibody is a dual-specificity that binds simultaneously to SORLA and HER2.

One aspect of the present invention relates to the medicinal use at least one SORL1 inhibiting agent for treating HER2-dependent cancer. This aspect may be formulated e.g. as a use of at least one SORL1 inhibiting agent for the manufacture of a medicament for use in treating a HER2-dependent cancer, or as a method of treating HER2-dependent cancer in a subject in need thereof by administering an efficient amount of at least one SORL1 inhibiting agent.

As used herein, the term “HER2-dependent cancer” refers to cancers that harbor either HER2 amplification in the genome or HER2 activating mutation and are addicted to HER2 function.

As used herein the term “HER2 amplification” refers to the amplification of the HER2 gene itself.

As used herein, the term “HER2 activating mutation” refers to a situation where the copy number of HER2 gene is normal, but due to an activating mutation cells express HER2 which is constitutively active.

Non-limiting examples of HER2-dependent cancers include breast cancer, lung adenocarcinoma, bladder cancer and ovarian cancer.

As used herein, the term “subject” refers to an animal, preferably to a mammal, more preferably to a human. Herein, the terms “human subject”, “patient” and “individual” are interchangeable.

As used herein, the term “treatment” or “treating” refers not only to complete cure of a disease, but also to alleviation, and amelioration of a disease or symptoms related thereto.

In the present invention, it was also realized that SORL1 inhibition sensitizes HER2-dependent cells to “cationic amphiphilic drugs” (CADs) which term refers to drugs that share several common physiochemical properties: hydrophobic ring structure on the molecule and a hydrophilic side chain with a charged cationic amine group, hence the class term cationic amphiphilic drugs (CADs).

In some further embodiments, SORL1 inhibiting agent may be used in combination with conventional “HER2-targeting drugs”, which term refers to any targeted therapy (including but not limited to antibodies: trastuzumab, pertuzumab; small molecule kinase inhibitors: lapatinib, afatinib, neratinib; antibody-drug conjugate: ado-trastuzumab emtansine [T-DM1]) for HER2 inhibition. It has been shown herein that SORL1 inhibiting agents sensitize cells to HER2-targeting drugs.

Thus, in some embodiments, the therapeutic aspect of the invention involves combined use of at least one SORL1 inhibiting agent and at least one drug selected from group consisting of CADs and/or HER2-targeting drugs. Efficient amounts of these substances may be provided or administered separately or in combination. For separate use, said at least one SORL1 inhibiting agent and said at least one drug may be administered simultaneously or subsequently.

In some further embodiments of the therapeutic aspect of the invention, at least one SORL1 inhibiting agent may be used in combination with at least one form of conventional cancer therapy, including chemotherapy, radiation therapy and immunotherapy.

As used herein, the term “efficient amount” refers to an amount in which the harmful effects of HER2-dependent cancer are, at a minimum, ameliorated. Amounts and regimens for the administration of the therapeutic agent can be determined readily by those with ordinary skill in the clinical art of treating cancer-related disorders. Generally, the dosage of the therapeutic agent depend on considerations such as: age, gender and general health of the patient to be treated; kind of concurrent treatment, if any; frequency of treatment and nature of the effect desired; extent of tissue damage; duration of the symptoms; and other variables to be adjusted by the individual physician. A desired dose can be administered in one or more applications to obtain the desired results. Pharmaceutical compositions according to the present embodiments may be provided in unit dosage forms.

For example, RNAi molecules may be administered in an effective amount within the dosage range of about 0.01 μg/kg to about 10 mg/kg, or the dosage range of about 1.0 μg/kg to about 10 μg/kg. RNAi molecules may be administered in a single daily dose, or the total daily dosage may be administered in divided doses, e.g. of two, three or four times daily.

As another non-limiting example, anti-SORLA antibodies are administered or used intravascularly at intervals ranging between once weekly to once every three months at doses in the range of 0.01 to 20 mg/kg, more preferably in the range of 0.1 to 10 mg/kg, most preferably 0.5 to 5 mg/kg. Alternatively, anti-SORLA antibodies may be administered or used subcutaneously at intervals ranging between once weekly to once every three months at doses in the range of 0.1 to 20 mg/kg, more preferably in the range of 0.2 to 10 mg/kg, most preferably 0.5 to 5 mg/kg.

In accordance with the above, one aspect of the invention relates to a pharmaceutical composition. Said pharmaceutical composition comprises at least one SORL1 inhibiting agent but it may further comprise at least one drug selected from CADs and HER2-targeting drugs.

For the purposes of parenteral or topical administration, the present therapeutic agent may be formulated, for instance, as solutions, suspensions or emulsions. The formulations may comprise aqueous or non-aqueous solvents, co-solvents, solubilizers, dispersing or wetting agents, suspending agents and/or viscosity agents, as needed. Non-limiting examples of non-aqueous solvents are propylene glycol, polyethylene glycol, vegetable oil, fish oil, and injectable organic esters. Fluid carriers include, for instance, water, water-alcohol solutions, including saline and buffered medial parenteral vehicles including sodium chloride solution, Ringer's dextrose solution, dextrose plus sodium chloride solution, Ringer's solution containing lactose, or fixed oils. Non-limiting examples of intravenous vehicles include fluid and nutrient replenishers, electrolyte replenishers, such as those based on Ringer's dextrose and the like. Aqueous compositions may comprise suitable buffer agents, such as sodium and potassium phosphates, citrate, acetate, carbonate or glycine buffers depending on the targeted pH-range. The use of sodium chloride as a tonicity adjuster is also useful. The compositions may also include other excipients, such as stabilizing agents or preservatives. Useful stabilizing excipients include surfactants (polysorbate 20 & 80, poloxamer 407), polymers (polyethylene glycols, povidones), carbohydrates (sucrose, mannitol, glucose, lactose), alcohols (sorbitol, glycerol propylene glycol, ethylene glycol), suitable proteins (albumin), suitable amino acids (glycine, glutamic acid), fatty acids (ethanolamine), antioxidants (ascorbic acid, cysteine etc.), chelating agents (EDTA salts, histidine, aspartic acid) or metal ions (Ca, Ni, Mg, Mn). Among useful preservative agents are benzyl alcohol, chlorbutanol, benzalkonium chloride and possibly parabens.

Solid dosage forms for oral administration include, but are not limited to, capsules, tablets, pills, troches, lozenges, powders and granules. In such solid dosage forms, dsRNAs and/or compounds of formula (I) may be admixed with at least one inert diluent such as sucrose, lactose or starch. Such dosage forms may also comprise, as is normal practice, pharmaceutical adjuvant substances, e.g. stearate lubricating agents or flavouring agents. Solid oral preparations can also be prepared with enteric or other coatings which modulate release of the active ingredients.

Non-limiting examples of liquid dosage forms for oral administration include pharmaceutically acceptable emulsions, solutions, suspensions, syrups and elixirs containing inert non-toxic diluents commonly used in the art, such as water and alcohol. Such compositions may also comprise adjuvants, such as wetting agents, buffers, emulsifying, suspending, sweetening and flavouring agents.

The pharmaceutical composition may be provided in a concentrated form or in a form of a powder to be reconstituted on demand. In case of lyophilizing, certain cryoprotectants are preferred, including polymers (povidones, polyethylene glycol, dextran), sugars (sucrose, glucose, lactose), amino acids (glycine, arginine, glutamic acid) and albumin. If solution for reconstitution is added to the packaging, it may consist e.g., of sterile water for injection or sodium chloride solution or dextrose or glucose solutions.

Means and methods for formulating the present pharmaceutical preparations are known to persons skilled in the art, and may be manufactured in a manner which is in itself known, for example, by means of conventional mixing, granulating, dissolving, lyophilizing or similar processes.

### EXAMPLES

### Example 1

SORLA is Highly Expressed in HER2 Amplified Breast Cancer Cell Lines and Correlates with HER2 in Bladder Cancer Patients

### Materials and Methods

### Cell Lines and Cell Culture

MDA-MB-361 cells were obtained from ATCC and grown in Dulbecco's modified essential medium (DMEM, Sigma) supplemented with 20% fetal bovine serum (FBS, Gibco), 1% vol/vol Pen/Strep (P0781-100ML; Sigma-Aldrich) and L-glutamine. BT474 and 5637 cells were obtained from ATCC and grown in RPMI1640 (Sigma-Aldrich) supplemented with 10% FBS, 1% vol/vol Pen/Strep and L-glutamine. JIMT-1 (obtained from DMZK), HCC1954 (ATCC), HCC1419 (ATCC), MCF7 (ATCC), MDA-MB-231 (ATCC), MDA-MB-436 (ATCC) and MFM-223 (ATCC) were grown in DMEM supplemented with 10% FBS, 1% Pen/Strep and L-glutamine. MCF10A (ATCC) and MCF10A DCIS.com (provided by J. F. Marshall, Barts Cancer Institute, Queen Mary University of London, London, England, UK) were grown in DMEM/F12 (Invitrogen, #11330-032) supplemented with 5% horse serum (Invitrogen#16050-122), 20 ng/ml human epidermal growth factor (E9644; Sigma-Aldrich), 0.5 mg/ml hydrocortisone (H0888-1G; Sigma-Aldrich), 100 ng/ml Insulin (19278-5ML; Sigma-Aldrich) and 1% vol/vol Pen/Strep. Cells were grown until 70-80% confluency before detaching and plating into new dish. Media was changed every three days.

### Western Blot Analysis

Protein extracts were sonicated (0.5 min ON/0.5 min OFF totally 5 min with full power) and protein levels were measured by Bio-Rad protein quantification kit. Sample buffer was added and samples were boiled for 5 min at 95° C. heat block. Proteins were separated on 4-20% gradient gel (Bio-Rad) and transferred into nitrocellulose membranes (Bio-Rad) by semi-dry turbo blot (Bio-Rad). Membranes were blocked with 5% milk powder in TBST for 1 hour at room temperature. Primary antibodies were diluted in blocking buffer (ThermoScientific) and PBS (1:1 ratio) mix and incubated over night at +4° C. Following primary antibodies were used: HER2 (Thermo Scientific, MA5-14057), SORLA (BD Transduction Lab, 612633), α-tubulin (Hybridoma Bank).

### Results

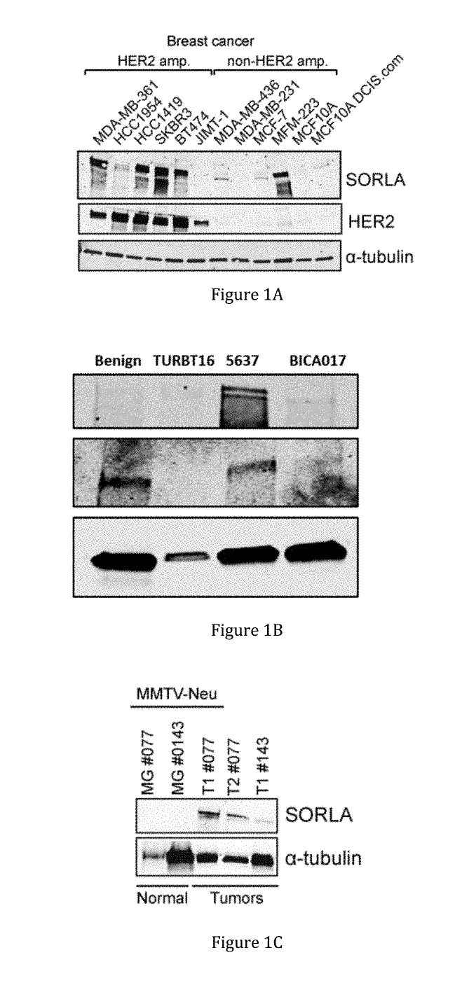

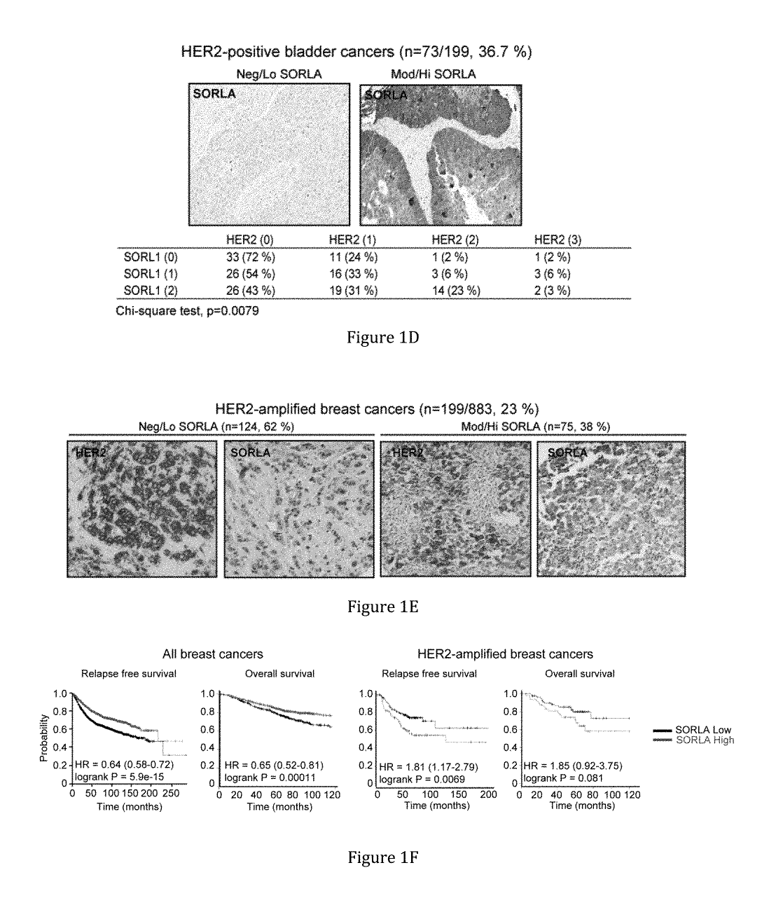

First we investigated expression of SORLA in different breast cancer cell lines. Western blot analysis of SORLA protein levels revealed that SORLA was mainly expressed in HER2 amplified breast cancer cell lines (FIG. 1A). In addition to breast cancer, SORLA was highly expressed in 5637 bladder cancer cell line with HER2 activating mutation (S310F) and overexpression when compared to HER2 negative T24 cell line and primary patient derived bladder cancer cells (FIG. 1B). We also detected elevated SORLA in MMTV-Neu tumors (FIG. 1C), indicating that SORLA is also expressed in a well-established mouse model of HER2.

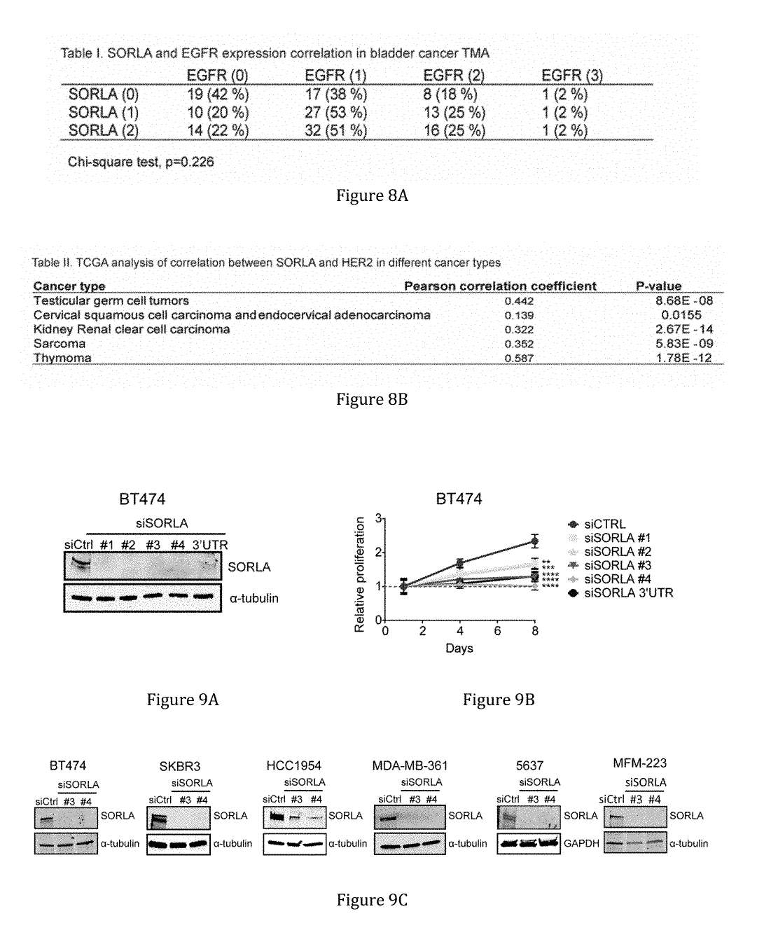

These in vitro findings indicative of a functional correlation between HER2 signalling and SORLA levels prompted us to investigate SORLA expression and possible correlation between HER2 and SORLA in clinical specimens. Immune histological staining of SORLA revealed that 78% of HER2 positive bladder carcinomas and 38% of HER2 amplified breast carcinomas expressed SORLA (FIGS. 1D and E). In bladder cancer HER2 and SORLA levels correlated significantly (Chi-square test. p=0.0079), whereas there were no correlation between SORLA and EGFR (FIG. 8A). On the other hand, in breast cancer no such correlation was found between SORLA and HER2 (FIG. 1E). However, in breast cancer a substantial proportion (38%) HER2 amplified breast cancers expressed moderate to high SORLA, indicating that HER2 amplified breast cancers fall into two subtypes with respect to SORLA positivity (FIG. 1E).

To study the link between HER2 and SORLA expression in other cancer types, we utilized the TCGA database and found a significant correlation between ErbB2 and SORL1 expression in testicular germ cell tumors, cervical squamous cell carcinoma and endocervical adenocarcinoma, kidney renal clear cell carcinoma, sarcoma and thymoma (FIG. 8B). Furthermore, in silico biomarker assessment tool (km-plot.com) that utilizes large data sets, such as TCGA, EGA and GEO, showed that high SORLA levels predict poor relapse free and overall survival specifically within HER2 amplified breast cancer patients (FIG. 1F). These together suggest that SORLA could play a previously unappreciated important role in HER2 dependent tumors.

### Example 2

### SORLA Regulates In Vitro Proliferation of HER2-Dependent Cancer Cells

### Materials and Methods

### Subcutaneous In Vivo Model

For subcutaneous (s.c.) tumors, 2 million siCTRL and siSORLA #3 5637 bladder cancer cells were injected s.c. in 100 μl (50% Matrigel) at the flank of 6 weeks old Nude mice. Mice were sacrificed after 29 days, and tumors were dissected, fixed in 10% formalin, and processed for paraffin sections with standard protocols. Sections were stained with hematoxylin-eosin (HE) and immunohistochemistry (IHC) for proliferation (Ki67) and apoptosis (TUNEL) markers.

All animal studies were ethically performed and authorised by the National Animal Experiment Board and in accordance with The Finnish Act on Animal Experimentation (Animal licence number ESAVI-9339-04.10.07-2016).

### Ductal Carcinoma In Situ (DCIS) Engraftment In Vivo Experiment

For intraductal tumor transplantation, MDA-MB-361 shCTRL and shSORLA breast cancer cells were resuspended in PBS as single cells (25 000 cells/ul). Trypan blue (0.1%) was added to the cell solution to visualize successful injection. Eight to ten weeks old female NOD scid mice were medicated with Temgesic for analgesia and anesthetized with isoflurane. After removal of abdominal hair, the tit of the abdominal (4^{th}) mammary glands was carefully snipped, and 4 μl (100 000 cells) of cell suspension was injected into the mammary ducts. A 30G Hamilton syringe with 50-μ1 capacity and a blunt-ended needle was used for the injection under a streomicroscope. Post-operation, the mice were further dosed with Rimadyl for extended pain relief. Mice were sacrificed 10 weeks after tumor inoculation, and abdominal mammary glands were dissected. Mammary glands were placed on an object glass, left to adhere for few minutes, and fixed in Carnoy's medium (60% EtOH, 30% chloroform, 10% glacial acetic acid) overnight (o/n) at +4° C. After rehydration in decreasing EtOH series and staining with carmine alum (0:2% carmine, 0.5% aluminium potassium sulphate dodecahydrate) o/n at room temperature (RT), samples were dehydrated and cleared in xylene for 2-3 days. Samples were mounted in DPX Mountant for histology (Sigma) and images were taken with Zeiss SteREO Lumar V12 stereomicroscope (NeoLumar 0.8× objective, Zeiss AxioCam ICc3 colour camera). All images per gland were combined automatically into a mosaic picture with PhotoShop. DCIS was quantified in ImageJ.

All animal studies were ethically performed and authorised by the National Animal Experiment Board and in accordance with The Finnish Act on Animal Experimentation (Animal licence number ESAVI-9339-04.10.07-2016).

### CAM Assay

Fertilized chicken eggs were incubated as previously described. Shortly, the eggs were washed and the development was started by placing the eggs in 37° C. incubator. On day 3 of development, a small hole was made in the eggshell to drop the CAM. On developmental day 10, a plastic ring was placed on the CAM and one million either control or SORLA targeting siRNA-transfected BT474 cells were implanted inside the ring in 20 μl of 50% Matrigel. After 6 days, tumors were imaged and dissected. The weight of dissected tumors were measured.

### Proliferation Assay

Cells were plated on 96-well plate (3000 cells/well, 4 replicates per sample) in volume of 100 μl. After 1 d, 4 d and 8 d of cell growth WST-8 reagent (Sigma, 96992) was added 10 μl/well and absorbance 450 nm was measured by plate reader after 1-2 h of incubation at 37° C. with 5% CO₂. Medium without cells was used as background and the A450 of background was subtracted from the samples. Relative proliferation was calculated by normalizing the A450 values of 4 d and 8 d to 1 d A450 values.

### Soft Agar Assay

Bottom agar (1.2%) in normal media was casted at the bottom of 12 well plates. Top agar (0.4%) in normal media was prepared and BT474 shCTRL or BT474 shSORLA cells were suspended in concentration of 20 000 cells/1.5 ml. Top agar (1.5 ml) with the cells were seeded on top of bottom agar (20 000 cells/well). Plate was shortly incubated at +4°C. to accelerate the solidification of top agar to maintain the single cell suspension and avoid the cells to drop down to the border of top and bottom agar. After top agar was solidified, 1 ml of normal medium was added on top. Medium was changed once a week. Soft agar colony size was measured after 5 weeks of growth by imaging the GFP signal by fluorescent microscope and measuring the area of GFP positive colonies by Image J.

Transient siRNA Transfections

Transfection of siRNAs to silence SORLA or HER2 protein expression: Cells were seeded on 6-well plate day before the transfection (300 000-500 000 cells/well). On next day, transfection mix containing siRNA (20-40 nM of targeting SORLA, 40 nM of HER2 targeting siRNA and 20-40 nM scramble siRNA) and RNAiMAX transfection reagent (Manufacturer) in OptiMEM medium was prepared. The mix was allowed to stand for 15 min at room temperature before adding to the cells on 6 well plate. After 48 hours of transfection cells were used for experiments.

### Colony Formation Assay

MDA-MB-361 and BT474 expressing either scramble shRNA or shRNA against SORLA (two individual) were plated on 6-well plate (1000 cells/well). Medium was changed twice a week and after 4 weeks of growth colony number was first measured by manual counting of colonies with more than 10 cells. After counting, the cells were stained with 0.2% Crystal Violet in 10% EtOH for 10 min at room temperature and washed with PBS.

### Cell Cycle Analysis

BT474 transfected with control siRNA and with two individual siRNA against SORLA (#3 and #4) and BT474 treated with DMSO or with 0.5 μM Lapatinib for 24 hours were trypsinized, spun down and suspended in ice cold PBS (1 ml). Cells were fixed with 70% EtOH (ice cold) by adding 3 ml of 70% EtOH in dropwise manner while gently vortexin the cells. Cells were then stored at +4° C. until propidium iodide (PI) staining was performed. PI (Sigma) staining was performed by preparing 25 μg/ml PI solution in PBS with RNAase A (Qiagen). Cells were washed twice with PBS. After that cells were suspended in PI solution and incubated 10 minutes on ice. Samples were protected from light and kept on ice until flow cytometry analysis. Samples were run with LSR II and cell cycle analysis was performed with FlowJo.

### Results

### SORLA Regulates In Vitro Proliferation of HER2-Dependent Cancer Cells

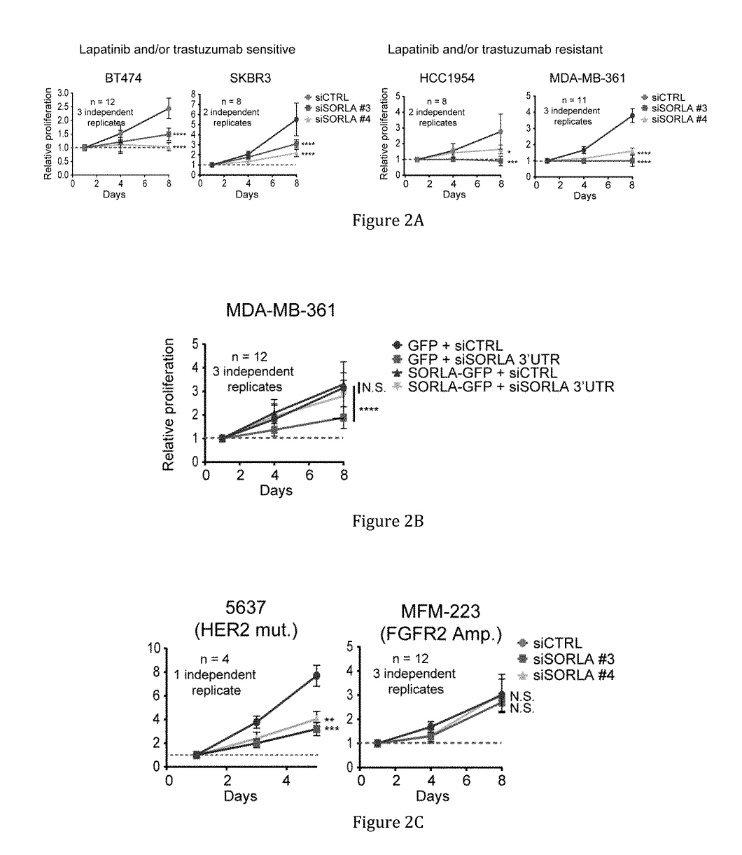

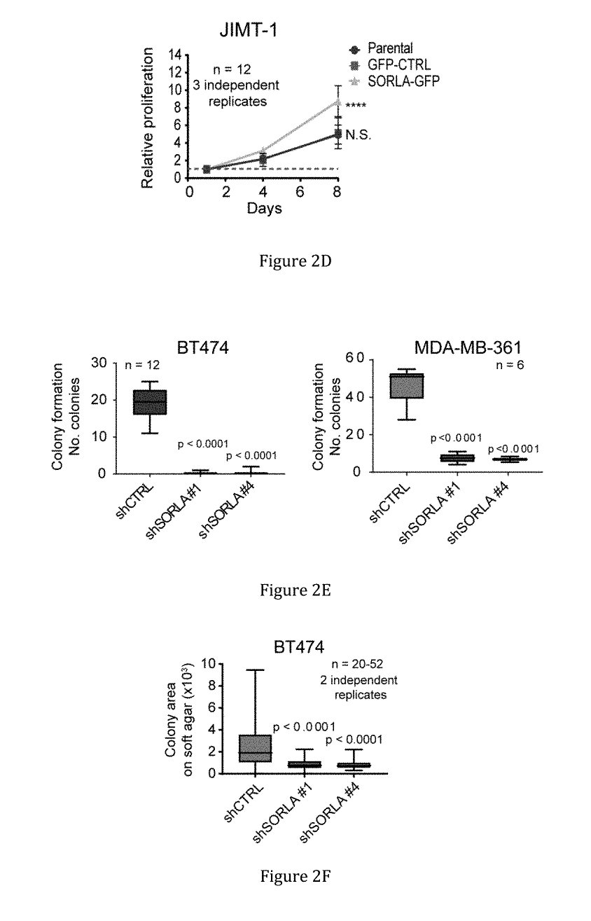

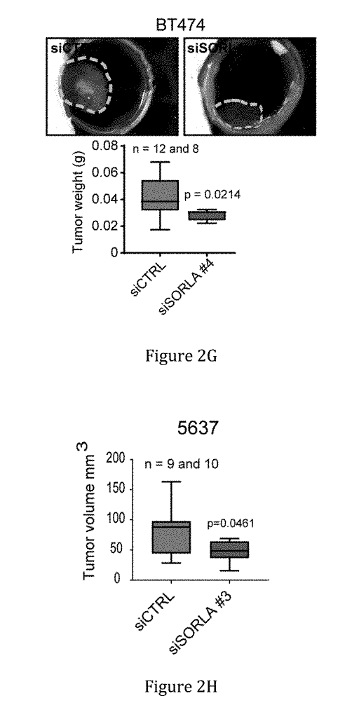

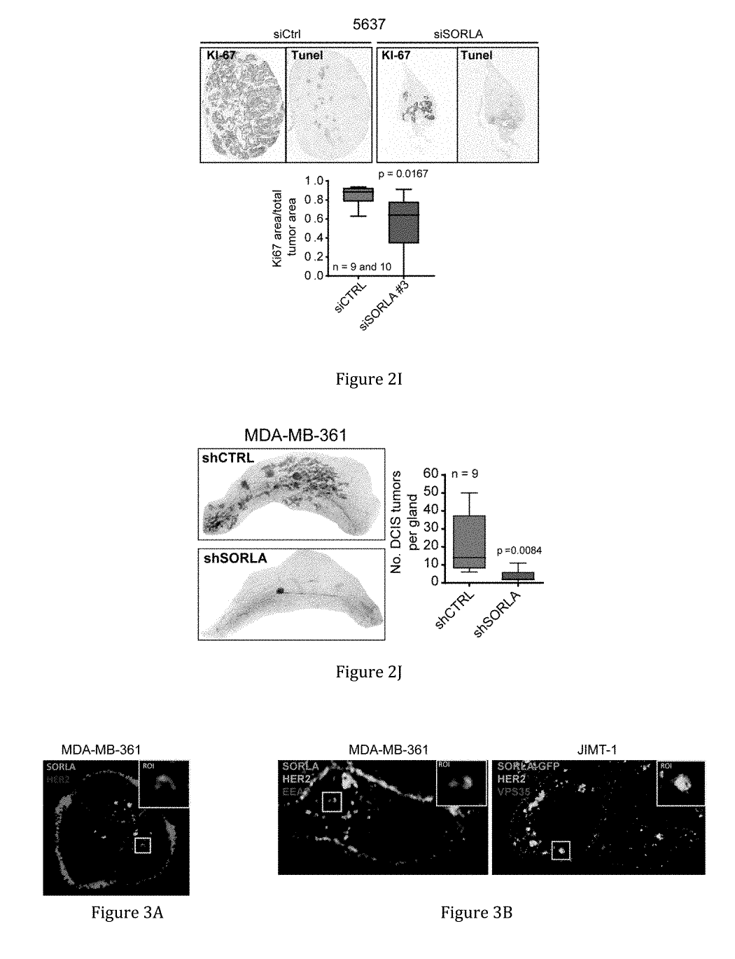

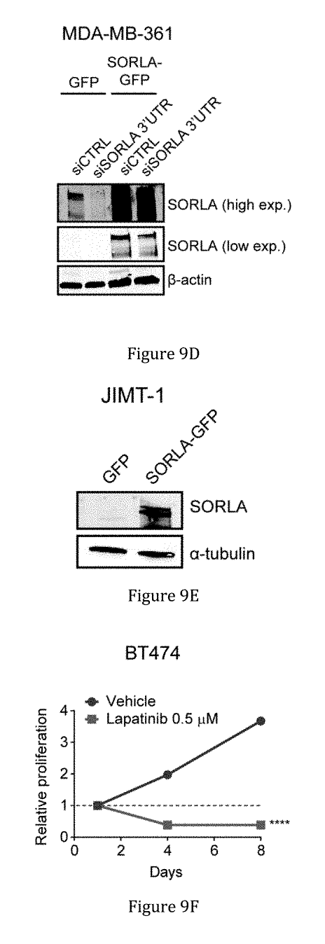

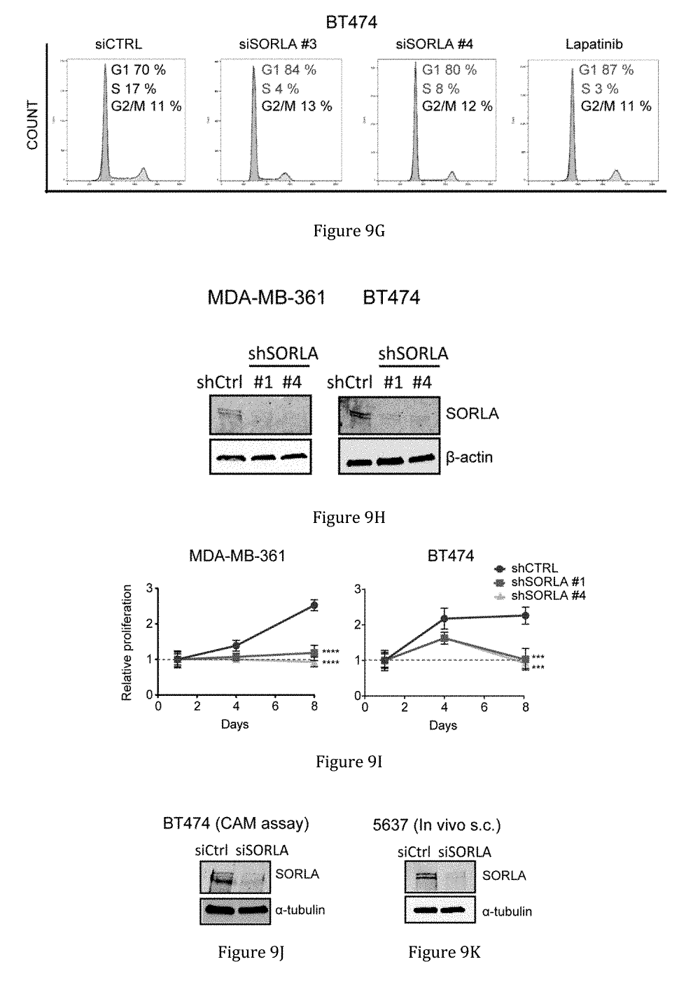

To explore whether SORLA plays a functional role in breast cancer cells, we silenced SORLA in two trastuzumab- and/or lapatinib-sensitive (BT474 and SKBR3; indicated in blue) and two trastuzumab- and/or lapatinib-resistant (MDA-MB-361 and HCC1954; indicated in pink) HER2-amplified breast cancer cell lines with endogenous SORLA expression. Initial validation was performed with five individual siRNAs against SORLA in BT474. All of the siRNAs efficiently silenced SORLA and interestingly significantly reduced cell proliferation (FIGS. 9A and 9B). In the other cell lines, silencing of SORLA with two independent siR-NAs (FIG. 9C) significantly reduced proliferation irrespective of their HER2 therapy resistance (FIG. 2A). Expression of SORLA-GFP in MDA-MB-361 cells silenced with siRNA targeting the 3′UTR of SORL1 mRNA fully restored cell proliferation to control levels (FIG. 2B; FIG. 9D) confirming that reduced proliferation of SORLA-silenced cells is specifically due to loss of SORLA and not triggered by off-target effects. The requirement for SORLA for HER2-dependent cancer cell proliferation was not restricted to breast cancer. The SORLA-expressing bladder cancer cell line 5637, with a HER2 activating mutation (S310F), was also sensitive to SORLA depletion (de Martino et al., 2014) (FIG. 2C; S2C).

To determine if the link between SORLA and cell proliferation was a specific trait of HER2-driven cancer cell lines, we sought to identify a SORLA expressing cell line with another oncogenic driver. We found that FGFR2-amplified MFM-223 breast cancer cells were highly positive for SORLA (FIG. 1A). Efficient silencing of SORLA with two siRNAs (FIG. 9C) had no effect on the proliferation of these cells (FIG. 2C), suggesting that the role of SORLA in supporting cancer cell proliferation could be specifically linked to HER2. Interestingly, a widely used model of HER2-positive lapatinib-resistant breast cancer, JIMT-1 cell line, lacked SORLA expression (FIG. 1A, FIG. 9E). Expression of SORLA-GFP significantly increased proliferation in JIMT-1 cells in comparison to GFP transfected or parental JIMT-1 cells (FIG. 2D). To study the proliferation profile in more detail, we performed a cell cycle analysis after SORLA silencing in BT474 cells. As a control, we also treated cells with 0.5 μM lapatinib for 24 hours. Silencing of SORLA led to increased accumulation of cells at G1/G0 and decreased number of cells in S phase, mimicking the effects of lapatinib treatment on cell cycle and proliferation (FIG. 9F, FIG. 9G).

### Silencing SORLA Compromises In Vitro and In Vivo Tumorigenesis of Both Anti-HER2 Therapy Resistant and Sensitive Cell Lines

To test the tumorigenic properties of SORLA-silenced cells in vitro and in vivo we generated stable BT474 and MDA-MB-361 cell lines expressing short hairpin RNA (shRNA) targeting SORLA (shSORLA) or scramble shRNA (shCTRL). Two individual shRNAs targeting SORLA showed efficient SORLA silencing (FIG. 9H) and significantly reduced proliferation of BT474 (sensitive) and MDA-MB-361 (resistant) cell lines in concordance with the results obtained with siR-NAs (FIG. 9I). To evaluate oncogenic growth in vitro, shCTRL- and shSORLA-transduced BT474 and MDA-MB-361 cells were seeded in 2D colony formation assay. SORLA depletion significantly interfered with colony formation in both BT474 and MDA-MB-361 cells (FIG. 2E). To further study the in vitro tumorigenesis in 3D anchorage independent conditions, shCTRL and shSORLA BT474 and MDA-MB-361 were also seeded on soft agar and colony formation was assessed 5 weeks later. At this time point, BT474 shSORLA cells formed significantly smaller colonies on soft agar than shCTRL cells (FIG. 2F). MDA-MB-361 cells did not grow on soft agar making them incompatible with this assay.

These in vitro findings were further validated in more in-vivo-like conditions in ovo. SORLA-silenced BT474 cells gave rise to significantly smaller tumors after six days of growth when compared to control cells following seeding on chicken chorioallantoic membranes (CAM) (FIG. 2G, FIG. 9J). Next, we wanted to test if SORLA silencing also affects tumor growth in vivo using two distinct models of HER2 therapy sensitive and resistant cancers. Subcutaneous grafting of lapatinib-sensitive 5637 cells, following transient SORLA silencing (the strong anti-proliferative effect of shSORLA in vitro precluded generating sufficient numbers of stable silenced cells for in vivo experiments), in nude mice revealed that loss of SORLA results in significantly smaller tumors when compared to control tumors expressing scramble siRNA with mean tumor volumes of 47.7 mm³ and 78.7 mm³, respectively (p=0.0461, FIG. 2H). Furthermore, SORLA-silenced tumors showed decreased proliferation measured by Ki-67 (FIG. 2I). SORLA silencing efficiency was confirmed by western blot in cells plated in vitro in parallel to the subcutaneous injection (FIG. 9K). Silencing of SORLA also compromised the in vivo tumor engraftment of anti-HER2 therapy-resistant HER2-amplified breast cancer cells in a fully orthotropic model. MDA-MB-361 shSORLA cells injected into mammary ducts of nod scid mice were almost fully impaired in forming ductal carcinoma in situ (DCIS) within 10 weeks when compared to shCTRL cells (FIG. 2J). Taken together these data indicate that SORLA plays a pivotal role in proliferation and tumor formation capacity of both anti-HER2 therapy sensitive and insensitive breast cancer cells, but also in HER2 mutated bladder cancer cells.

### Example 3

SORLA Associates, and Co-Localizes, with HER2 on the Plasma Membrane, Early Endosomes and Retrograde Trafficking Vesicles

### Materials and Methods

### Co-Immunoprecipitations

SKBR3 cells were transiently transfected with pLenti-c-mGFP Empty vector (GFP-ctrl) and pLenti-c-mGFP SORLA (SORLA-GFP) vector. After 24 h of transfection, GFP-trap immunoprecipitation of GFP-fused SORLA protein or GFP only were performed according to manufacturer's protocol (Chromotek; gtak-20). After co-immunoprecipitation, lysates were separated on 4-20% gradient gel (BioRad) and transferred into nitrocellulose membrane (BioRAD) by turboblot. Membranes were blocked by 5% milk in TBST for 1 h at room temperature. Primary antibodies were incubated over night at +4° C. Primary antibodies used: Mouse anti-HER2 (ThermoScientific; MA5-14057) diluted 1:1000 in 5% milk in TBST and rabbit anti-GFP (Molecular Probes; A11122) diluted 1:1000 in 5% milk in TBST. After primary antibody incubation, membranes were washed three times with TBST for 15 min at room temperature and secondary antibodies were incubated 1 hour at room temperature. Secondary antibodies used: anti-mouse 800 and anti-rabbit 680. Secondary antibodies were washed off with TBST three times for 15 min at room temperature and membranes were scanned with Odyssey IR. Endogeneous HER2 pull-down in 5637 cells was performed as described below. Cells were detached with Trypsin/HyQTase and cells were spun down. Medium was removed and 200 μl of lysis buffer (40 mM HepesNAOH, 75 mM NaCl, 2 mM EDTA, 1% NP40, protease—and phosphatase inhibitor pills) per 10 cm dish was added. Lysates were moved to 1.5 ml tube and twirled 30 min at +4° C. rotator. Lysates were spun down with 13 000 rpm 10 min +4° C. Twenty microliter was taken from supernatant for lysis control, which was frozen immediately. Rest of the sample was moved to new 1.5 ml tubes and mouse anti-HER2 antibody (1 μg/sample; ThermoScientific; MA5-14057) was added. Tubes were twirled in rotator at +4° C. overnight. ProteinG-beads with cut tip 30 μl/sample were taken. Beads were spun 3000 rpm 3 min and ethanol was removed. Beads were washed two times with PBS (500 μl) and spun down with 3000 rpm 3 min to remove PBS. Beads were blocked with 0.5% BSA-PBS buffer overnight (at +4° C. rotator). In the next day beads were added to the lysis and twirled in rotator (+4° C.) 1 hour. After that, beads were wash 3 times with wash-buffer (20 mM Tris-HCl (pH 7.5), 150 mM NaCl, 1% NP-40; 500 μl) and were spun down with 3000 rpm 3 min. Finally, sample buffer was added and samples were run on western as described above. Primary antibodies were incubated over night at +4° C.: mouse anti-SORL1 (BD Transduction Lab; 612633) 1:1000 in 5% milk in TBST and mouse anti-HER2 (ThermoScientific; MA5-14057) 1:1000 in 5% milk in TBST. Primary antibodies were washed off with TBST (three times) and secondary antibodies were incubated 1 hour at room temperature: anti-mouse 800 1:1000 in 5% milk in TBST. Secondary antibodies were washed off eith TBST (three times) and signal was detected by Odyssey IR.

### Immunofluorescent Staining and Imaging

Cells were plated on ibidi 8-well dishes or in some cases in 3.5 mm ibidi dishes. Cells were fixed with 4% paraformaldehyde (PFA) in phosphate buffer saline (PBS) for 10 min at room temperature. PFA was quenched by incubating with 50 mM NH4Cl for 15 min at room temperature. Cells were blocked and permeabilized with 30% horse serum in PBS +0.3% Triton X-100 15 min at room temperature. Primary antibodies were diluted in 30% horse serum and incubated over night at +4° C. HER2 staining was performed with Herceptin (Roche) or with mouse monoclonal antibody (ThermoScientific; MA5-14057) in dilution of 1:300, LAMP1 antibody (Santa Cruz; SC-20011 (H4A3)) was diluted 1:50, rabbit monoclonal SORLA antibody (CM Petersen Lab, Arhus University) in dilution of 1:300, goat anti-EEA-1 (Santa Cruz; sc-6415) 1:50, goat anti-VPS35 (Abcam; ab10099) 1:300, mouse anti-CD63 (Hybridoma Bank; H5C6) 1:300 and rabbit anti-TGN46 (Abcam; ab50595) 1:300. After primary antibody incubation, cells were washed three times with PBS for 5 min at room temperature and then secondary antibodies were diluted 1:300 in 30% horse serum. Following secondary antibodies were used: anti-mouse AlexaFLuor 488 (Life Technologies), anti-rabbit (AlexaFluor 488 (Life Technologies), anti-human AlexaFluor 568 (Life Technologies), anti-goat AlexaFluor 647 (Life Technologies), anti-mouse AlexaFLuor (Life Technologies). Secondary antibodies were incubated 1 hour at room temperature together with DAPI (1:1000). Secondary antibodies and DAPI were washed off by PBS three times and samples were wither imaged right away or stored at +4° C. in dark until imaging. Imaging was performed wither with Zeiss LSM780 (Carl Zeiss Microscopy, Thornwood, N.Y.) or 3i spinning disk confocal (Marianas spinning disk imaging system with a Yokogawa CSU-W1 scanning unit on an inverted Carl Zeiss Axio Observer Z1 microscope, Intelligent Imaging Innovations, Inc., Denver, USA.)

### Live-Cell Imaging of SORLA-GFP and Tz-568 in MDA-MB-361 Cells

The cells were kept on ice and washed twice with ice cold PBS. 567 alexa fluor labelled Herceptin was diluted (1:200 from 30 ug/ml) in Hank's Balanced Salt Solution and put on cell for 1 hr away from light. The cells were then again washed with ice cold PBS twice and finally warm serumless media (respective media for cell type) supplemented with 5% HEPES was added to the cells.

### Results

SORLA Associates, and Co-Localizes, with HER2 on the Plasma Membrane, Early Endosomes and Retrograde Trafficking Vesicles

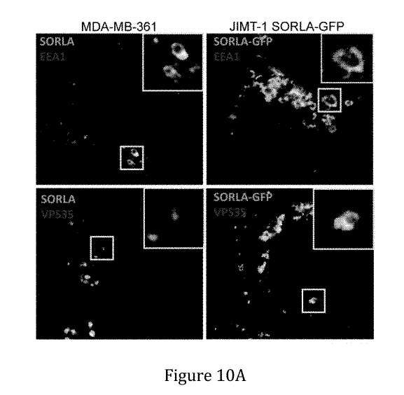

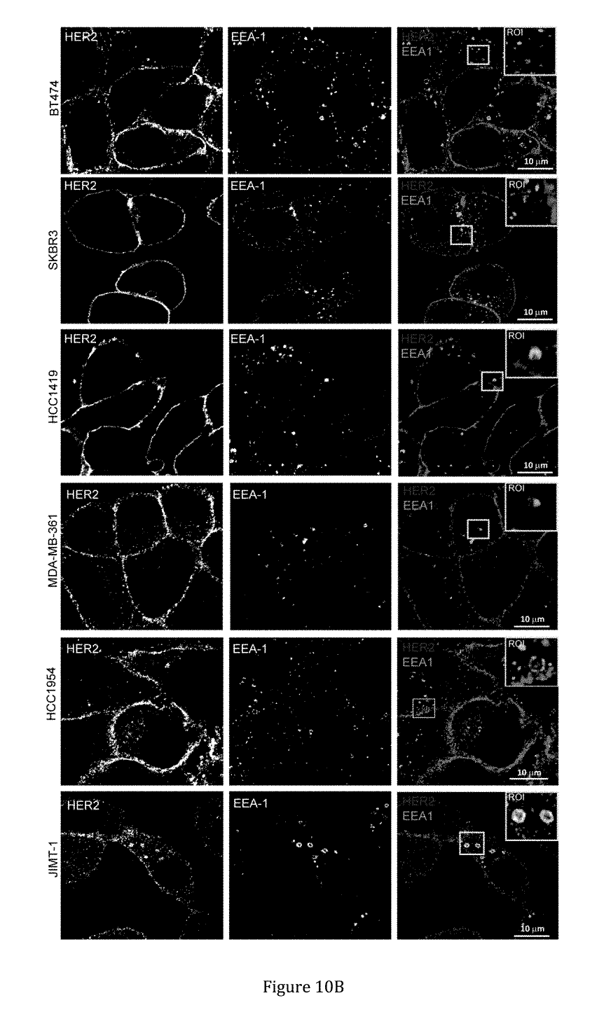

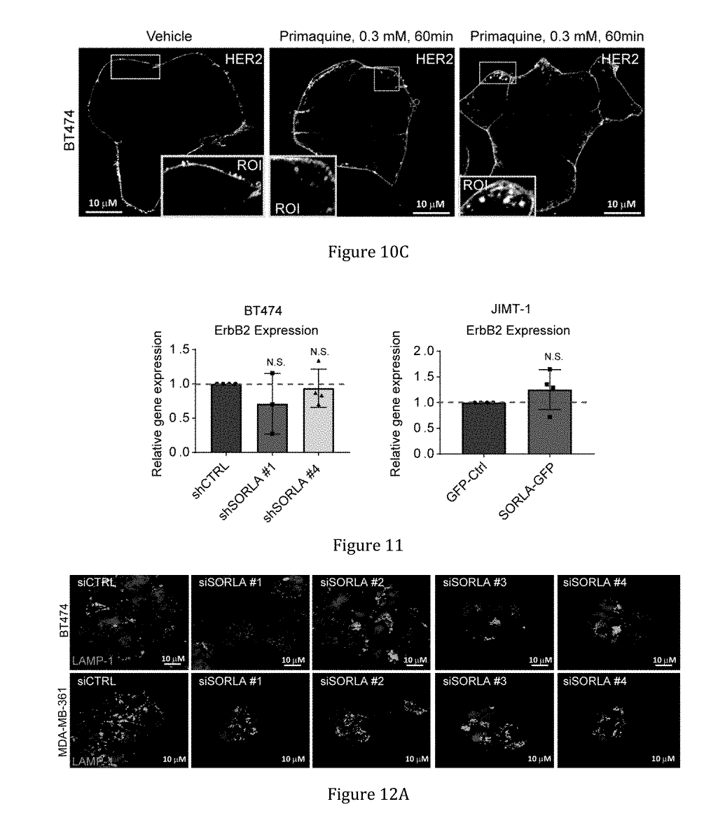

SORLA has previously been implicated in the traffic of cell surface proteins APP and IR in neuronal cells and adipocytes respectively (Andersen et al., 2005; Andersen et al., 2006; Schmidt et al., 2016). As SORLA has not previously been studied in cancer, we performed a panel of co-transfections with SORLA-GFP and markers of different endosomal compartments to investigate the subcellular localization of SORLA. SORLA was found to localize largely to early endosomes (EEA-1) and retrograde vesicles (VPS35) (FIG. 10A). Previous studies have indicated that HER2 is mainly restricted to the plasma membrane due lack of ligand-induced internalization (Bertelsen and Stang, 2014) and therefore subcellular traffic of HER2 has remained poorly studied compared to other oncogenic receptor tyrosine kinases such as EGFR or MET (Hammond et al., 2003; Henriksen et al., 2013; Sigismund et al., 2013). Since most of the previous studies are largely based on a very limited number of cell lines, namely SKBR3 or non-HER2-amplified cell lines with exogenous overexpression of HER2 (Austin et al., 2004; Baulida et al., 1996; Hommelgaard et al., 2004), we wanted to screen all our HER2-amplified cell lines in the context of endogenous HER2 localization. HER2 was mainly on the plasma membrane in SKBR3, in accordance with previous work (Austin et al., 2004; Hommelgaard et al., 2004), and did not overlap with EEA-1 (FIG. 10B). However, intracellular HER2 was readily detected in MDA-MB-361, JIMT-1 and HCC1954 cells with a proportion of the intracellular pool clearly overlapping with EEA-1 indicating localization into early endosomes (FIG. 10B). Moreover, in BT474 cells inhibition of vesicular recycling with primaquine resulted in accumulation of intracellular HER2, indicating that HER2 undergoes constant endocytosis balanced with very rapid recycling in these cells (FIG. 10C).

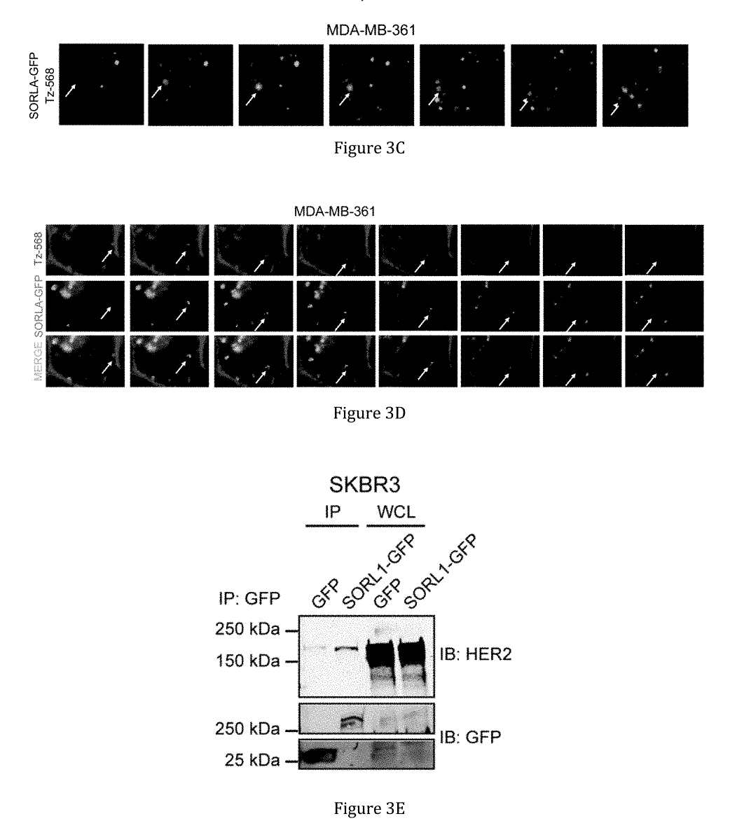

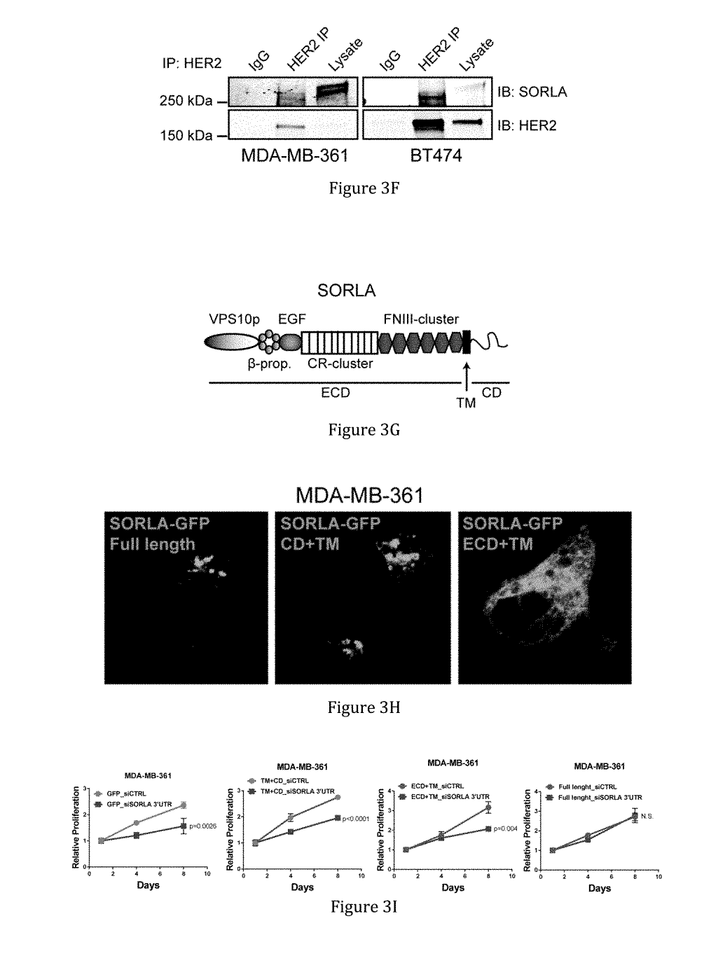

In MDA-MB-361 cells endogenous SORLA co-localizes with HER2 in endosome-resembling structures (FIG. 3A). These were either EEA-1 or VPS35 positive (FIG. 3B) suggesting that SORLA and HER2 may undergo co-trafficking between the plasma membrane and endosomes in these cells. To study the dynamic movement of SORLA and HER2 in detail, we performed live cell TIRF imaging (allowing visualization of events close to the plasma membrane) with MDA-MB-361 SORLA-GFP cells labelled with Alexa568 conjugated trastuzumab (Tz-568). Transient short-lived SORLA- and HER2-positive structures were detected in the TIRF-plane, indicative of active dynamics to and from the plasma membrane. In addition, co-localizing puncta were frequently observed undergoing dynamic lateral movement on the plasma membrane (FIG. 3C). Live-cell imaging of the intracellular part of the cell also showed that SORLA and HER2 move together within the same endosomal structures (FIG. 3D).

Intrigued by the apparent co-trafficking of SORLA and HER2, we next performed a set of immunoprecipitation assays to investigate whether HER2 and SORLA associate. SORLA-GFP co-precipitated endogenous HER2 from SKBR3 cells (FIG. 3E) and endogenous HER2 co-precipitated SORLA in MDA-MB-361 and BT474 cells, indicating that HER2 and SORLA are in the same complex (FIG. 3F). SORLA consists of an extracellular domain (ECD), a transmembrane domain (TM) and a short cytosolic domain (CD) (FIG. 3G). Importantly, either the HER2-associating ECD+TM or the correctly localizing TM+CD were sufficient to rescue the effect of SORLA silencing in MDA-MB-361 cells (FIGS. 3H and 3I), suggesting that association with HER2 and unperturbed trafficking are both requirements for SORLA-mediated proliferation of HER2-dependent cancer cells.

### Example 4

### SORLA Positively Regulates HER2 Plasma Membrane Levels

### Materials and Methods

### Analysis of Cell Surface HER2 Levels by Flow Cytometry

Cells were detached by HyQtase and spun down. Next, cells were fixed with 4% PFA in PBS for 15 min at room temperature and followed by washing with PBS (three times). Cells were stained with 1:200 dilution of rabbit anti-HER2 (9G6, Abcam; Ab16899) primary antibody for 1 hour at room temperature. Samples were washed with PBS and anti-rabbit AlexaFluor 647 (Life Technologies) secondary antibody was diluted 1:300 and incubated 1 h at room temperature. Cells were washed two times with PBS and analyzed by FACS calibur. Data analysis was performed with Flowing software.

RNA Extraction, cDNA Synthesis and qPCR

Cells were lysed in RA lysis buffer and RNA was extracted according to manufacturer's instructions (NucleoSpin RNA extraction kit, Macherey-Nagel, 740955.5). RNA concentrations were measured by NanoDrop. Complementary DNA (cDNA) was synthesized by high-capacity cDNA reverse transcription kit (Applied Biosystems) according to the manufacturer's protocol. Quantitative real time PCR reactions with TaqMan probes were performed according to manufacturer's instructions (Thermo/Applied Biosystems, TaqMan™ Universal Master Mix II, 4440040). Following TaqMan probes (ThermoScientific, 4331182) were used: TaqMan probe for LAMP1 (Hs00174766_m1), TaqMan probe for CTSD (Hs00157205_m1), TaqMan probe for HEXA (Hs00166843_m1), TaqMan probe for HEXA (Hs00166843_m1), TaqMan probe for ATP6V0E1 (Hs00748673_s1), TaqMan probe SORL1 (Hs00268342_m1), TaqMan probe for ErbB2 (Hs01001580_m1). Relative quantification of gene expressions values were calculated using the ddCt method (Genome Res. 1996 October; 6 (10):986-94.)

### Biotin-Based HER2 Endocytosis Assay

HER2 endocytosis was measured using a cell-surface biotinylation-based assay as previously described (Arjonen et al., 2012). Shortly, MDA-MB-361 siSORLA silenced and control silenced as well as JIMT-1 GFP-ctrl and SORLA-GFP cells were grown to 80% confluence, placed on ice, and washed once with cold PBS, and cell-surface proteins were labeled with 0.5 mg/ml of EZ-link cleavable sulfo-NHS-SS-biotin (#21331; Thermo Scientific) in Hanks' balanced salt solution (H9269; Sigma) for 30 min at 4° C. Unbound biotin was removed, and cells were washed with cold media and allowed to internalize receptors in prewarmed 10% serum-containing medium at 37° C. for the indicated times. Cells were then quickly placed back on ice with the addition of cold media. The remaining biotin at the cell surface was removed with 60 mM MesNa (63705; sodium 2-mercaptoethanesulfonate: Fluka) in MesNa buffer (50 mM Tris-HCl [pH 8.6], 100 mM NaCl) for 30 min at 4° C., followed by quenching with 100 mM iodoacetamide (IAA, Sigma) for 15 min on ice. To detect the total surface biotinylation, one of the cell dishes was left on ice after biotin labeling and did not undergo internalization or MesNa treatment. Cells were then washed with PBS, scraped in lysis buffer (50 mM Tris pH 7.5, 1.5% Triton X-100, 100 mM NaCl, 1 tablet phos-Top, 1 tablet complete EDTA) at 4° C. for 20 min. All cell extracts were cleared by centrifugation (14,000×g, 10 min, 4° C.), and biotinylated HER2 was immunoprecipitated from the supernatants with appropriate antibodies and protein G sepharose beads (17-0618-01; GE Healthcare).

Biotinylated internalized HER2 and total receptor levels were detected by immunoblotting with horseradish peroxidase (HRP)-conjugated anti-biotin antibody (#7075; Cell Signaling Technology) and receptor-specific antibodies, respectively.

Enhanced chemiluminescence-detected biotin and receptor signals were quantified as integrated densities of protein bands with ImageJ (v. 1.43u), and each biotin signal was normalized to the corresponding receptor and total biotin signal. The endocytosis rate of HER was similarly measured in MDA-MB-361 control- or SORLA-silenced cells and in JIMT-1 GFP-Ctrl and SORLA-GFP cells.

### Imaged Based Herceptin Internalization Assay

MDA-MB-361 SORLA (siSORLA #3) and scramble (siCTRL) silenced cells were plated plated on ibidi 35 mm μ-dishes after 72 hours of silencing. On the next day, cells were washed once with PBS and incubated with Trastuzumab conjugated with AlexaFluor568 (Tz-568) 1:200 in cold Hank's Balanced Salt Solution on ice protected from light for 1 hour. After 0 min, 15 min, 30 min and 60 min incubation at 37oC after addition of warm serum free media, cells were washed twice with cold PBS and fixed with 4% PFA for 10 minutes at room temperature. After that cells were washed twice with PBS and stored at +4° C. in dark until imaging with 3i spinning disk confocal microscope. Several fields were randomly imaged and intracellular Tz-568 signal was analysed from max intensity projections of 6 stacks taken from the middle plane of the cell (determined by DAPI signal). Intracellular signal was quantified with ImageJ by manually gating the intracellular part of the cell and not the plasma membrane Tz-568 signal. Results are pooled from two independent biological replicates.

### Results

### Silencing SORLA Leads to Decreased HER2 Protein Levels and Increased Internalization

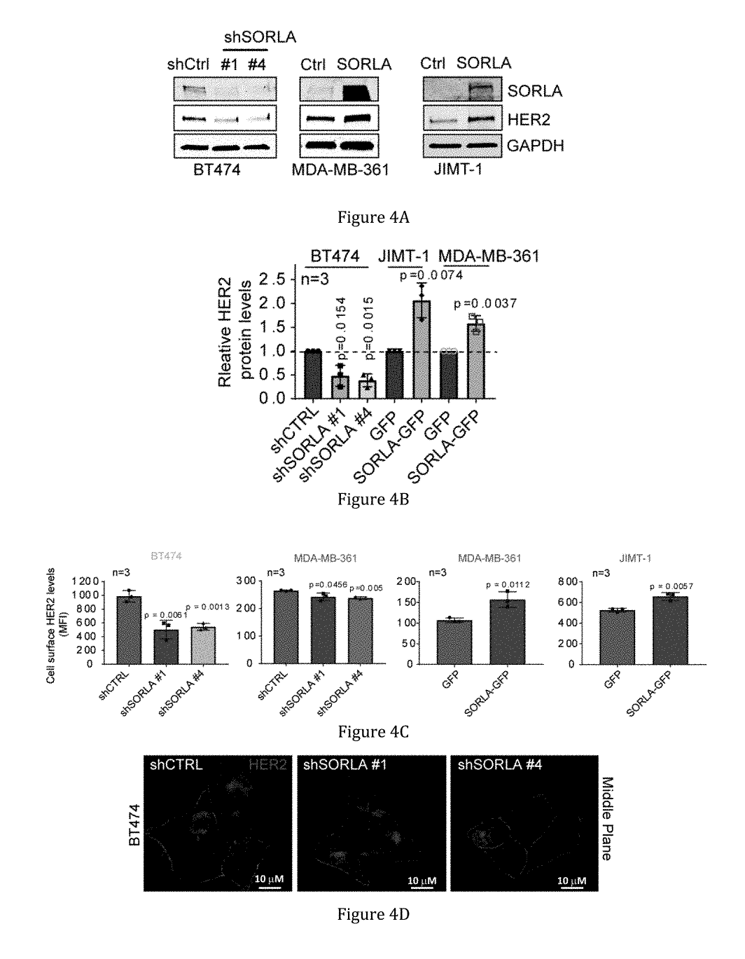

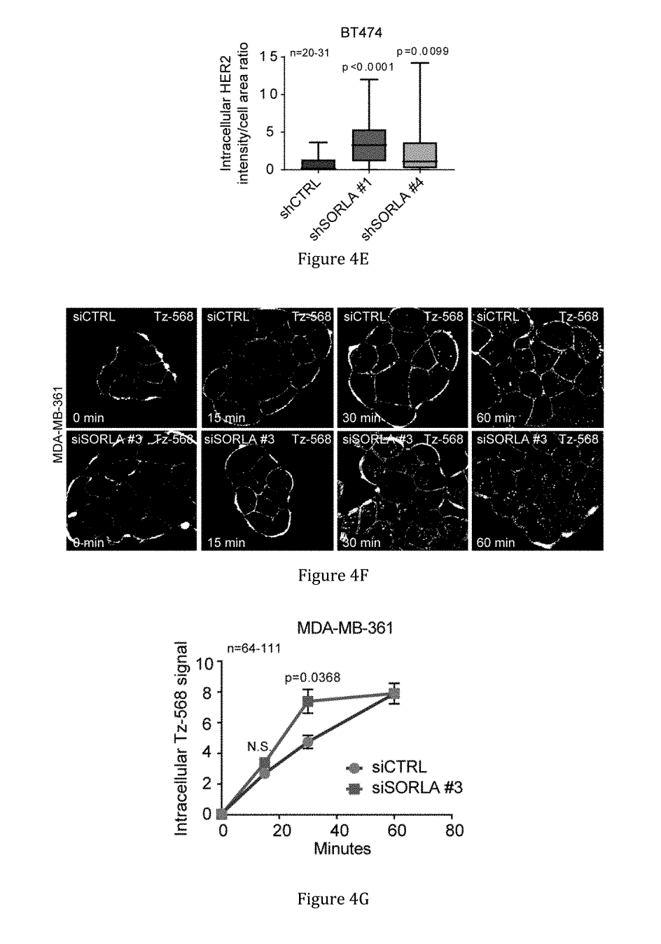

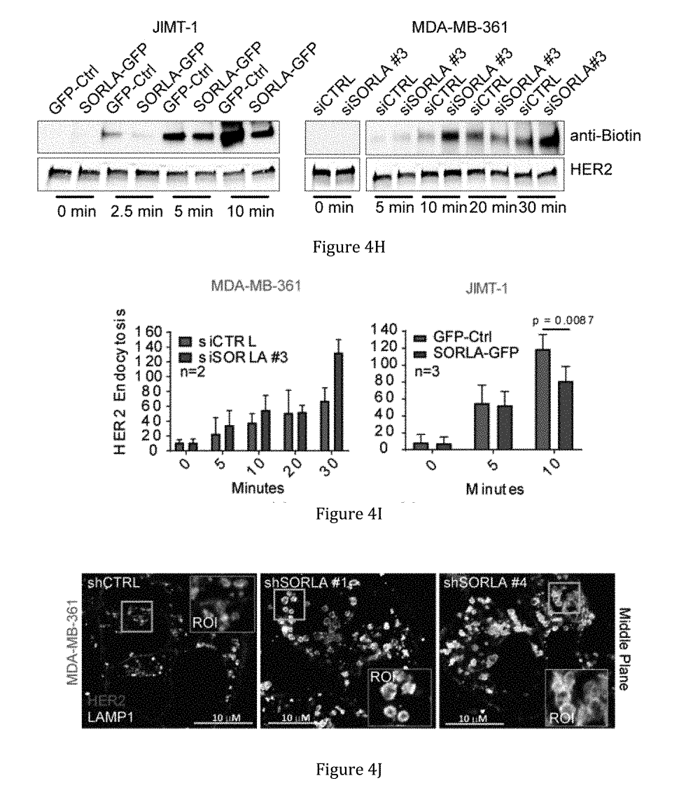

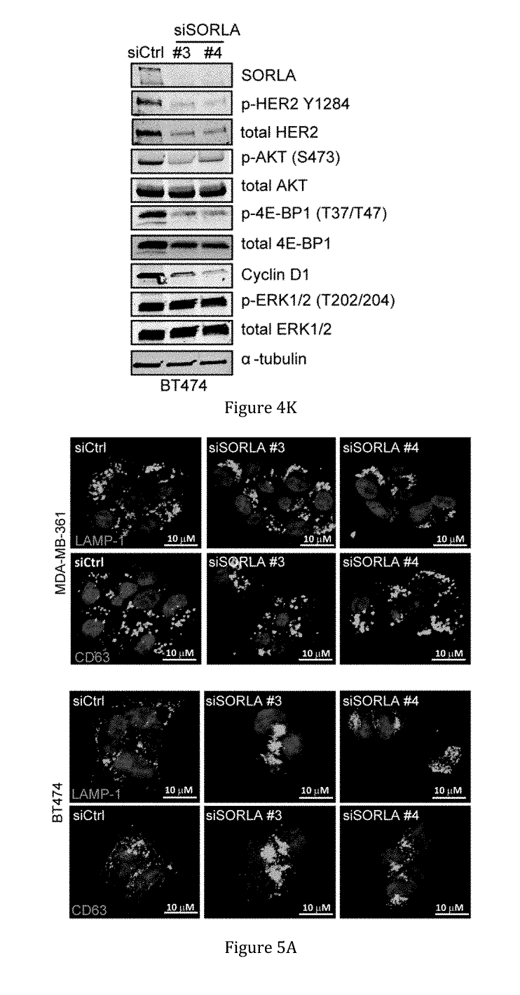

Since HER2 amplification is the major driver of proliferation and tumorigenesis in our cell models, we wanted to check if SORLA regulates HER2. Silencing of SORLA led to about 50% decrease in total HER2 protein levels in BT474 and MDA-MB-361 cells (FIGS. 4A and 4B). Conversely, overexpression of SORLA in MDA-MB-361 and JIMT-1 cells increased total HER2 protein levels by approximately 100% and 50%, respectively (FIGS. 4A and 4B). We also measured HER2 cell surface levels by flow cytometry after SORLA silencing or overexpression. SORLA silencing caused a modest but significant decrease in total HER2 cell surface levels in BT474 and MDA-MB-361 cells, whereas the overexpression of SORLA led to increased HER2 cell surface expression (FIG. 4C). qPCR analysis of ErbB2 mRNA levels after SORLA silencing or overexpression did not show any significant difference indicating that SORLA-mediated regulation of HER2 occurs at the protein level (FIG. 11). However, considering the very high HER2 expression in these receptor-amplified cell lines, the extent of HER2 down-regulation seemed insufficient to explain the strong anti-proliferative effect of SORLA depletion. Subcellular localization of receptors may strongly influence their downstream signalling capability (Sorkin and von Zastrow, 2009). To test the possibility that the absence of SORLA triggers HER2 mislocalization, we analyzed HER2 localization in shSORLA and shCTRL BT474 cells. Interestingly, SORLA silencing led to increased intracellular accumulation of HER2, normally not observed in these cells (FIGS. 4D and 4E). To study the internalization of HER2 in more detail, we used Alexa-568-conjugated trastuzumab (Tz-568) in an imaging-based endocytosis assay. SORLA was silenced in MDA-MB-361 cells and cell surface HER2 was labelled with Tz-568 on ice. After staining, the internalization of Tz-568 was induced by incubating cells at +37° C. for 15 min, 30 min, and 60 min before fixing. The amount of internalized Tz-568 was quantified from fixed cells. Interestingly, SORLA-silenced cells showed significantly faster internalization of HER2 (FIGS. 4F and 4G). Similar data was obtained with a biochemical endocytosis assay of unperturbed HER2, indicating that these effects are not secondary to trastuzumab-induced HER2 uptake (Austin et al., 2004). In MDA-MB-361 cells, silencing of SORLA led to increased HER2 endocytosis (FIGS. 4H and 4I), whereas the overexpression of SORLA in JIMT-1 cells inhibited HER2 uptake (FIGS. 4H and 4I). Concordant with the altered tz-568-labelled HER2 localization in BT474 cells with transient SORLA silencing, MDA-MB-361 cells treated with shSORLA displayed a striking accumulation of HER2 in LAMP-1 positive structures not observed in control cells (FIG. 4J). HER2 signaling along the PI3K/Akt axis is critical for HER2 growth promoting functions in cancer cells. The intracellular HER2 localization on late endosomes in shSORLA cells could influence HER2 downstream signaling. Silencing of SORLA in BT474 cells led to decreased phosphorylation of AKT (Ser473) and 4E-PB1 (Thr37/46) as well as decreased cyclin D1 levels. Interestingly, ERK½ (T202/204) phosphorylation remained unaffected (FIG. 4K), suggesting that SORLA silencing specifically influences P13K-dependent signaling in HER2-amplified cells.

### Example 5

### Silencing SORLA or Disrupting HER2 Signaling Leads to Dysfunctional Lysosomes

### Materials and Methods

### DQ Red BSA Assay

Protocol for DQ Red BSA (ThermoScientific, D12051) staining for imaging: Briefly, MDA-MB-361 cells were transiently transfected with two individual siRNAs against SORLA (siSORLA #3 and siSORLA #4) and with scramble (siCTRL) as described above. After 72 hours of silencing, cells were plated on ibidi 8-well μ-slide. On next day, DQ Red BSA 25 μg/ml solution was prepared by diluting it in warm medium. The DQ Red BSA medium was changed to cells on ibidi 8-well μ-slides (200 ul/well) and cells were incubate at 37° C. for 48 hours. After incubation, cells were washed twice with PBS and fixed with 4% PFA for 10 minutes at room temperature. Cells were finally washed two times with PBS and imaged with LSM780. Protocol for DQ Red BSA staining for flow cytometry: Briefly, MDA-MB-361 were silenced with SORLA targeting siRNAs and scramble as described above. After 72 hours of silencing cells were stained with DQ Red BSA 25 μg/ml solution by diluting it in warm medium. DQ Red BSA solution was added to cells (1 ml per well in 6-well plate). Cells were incubated at 37° C. for 24 hours. For the Bafilomycin control, Bafilomycin was added to a separate 25 μg/ml DQ Red BSA solution to make the final Bafilomycin concentration 25 nM and were incubated 4 h instead of 24 h. The DMSO control solution was prepared the same way as the Bafilomycin solution. After DQ Red BSA staining, cells were washed with PBS and detached by HyQtase for 5 min at 37° C. Detached cells were transferred to Eppendorf tubes and washed with PBS couple of times and finally fixed with 4% PFA for 15 min at room temperature. Cells were washed twiced with PBS and resuspend in 200 μl PBS. Samples were store at +4° C. protected from light few days before detecting the DQ Red BSA signal with XXX channel in LSRFortessa. Flow cytometry data was analysed by Flowing software. Unstained cells were used as background and it was subtracted from the DQ Red BSA stained samples. The DQ Red BSA signal from SORLA silenced cells (siSORLA #3 and siSORLA #4) was normalized to scramble silenced (siCTRL) cells. Similarly the signal from Bafilomycin treated cells was normalized to DMSO treated cells. Results are pooled from five independent biological replicates.

### Subcellular Fractionation

Control siRNA silenced and SORLA silenced BT474 cells (10 cm dish) were washed with PBS and scraped with 500 μL of hypotonic lysis buffer (10 mM HEPES-KOH pH 7.2, 0.25 M sucrose, 1 mM EDTA, 1 mM MgOAc and protease and phosphatase inhibitor pills (PhosSTOP and Complete from Roche). After that, cells were lysed with cell cracker multiple times and 40 μl of total lysate was saved. Rest of the lysate was centrifuged at 1000×g for 10 min to remove nucleus and cell debris. The supernatant was ultra-centrifuged at 100 000×g to collect total membrane fraction (pellet) and cytosolic fraction (supernatant). All fractionation steps were performed at 4° C. or on ice. Finally, the reducing Laemmli buffer was added to fractions and denatured for 5 min at 95° C. for western blotting.

### TFEB Localization Assay

SORLA was silenced with two individual siRNAs together with scramble siRNA as described above. After 48 hours of transfection, cells were seeded on 8-well ibidi dishes (20 000 cells/well). Transfection of pEGFP-N1-TFEB and pEGFP-N1-TFEB-S3A,R4A were performed as described below: In one tube the Lipofectamine 3000 was added in OptiMEM medium. In other tube plasmid (3 μg) was mixed with Lipofectamine 3000P reagent in OptiMEM medium. Tubes were mixed and allowed to stand for 15 min at room temperature. Meanwhile, Opti-MEM media was changed to cells on 8-well ibidi dish. Finally transfection mix was added to cells. After 24 hours of transfection, cells were washed with PBS and then either normal media was added to cells or some of the siCTRL cells transfected with wild type TFEB were starved with Hank's balanced salt solution for 3 hours. After 3 hours, cells were fixed with 4% PFA for 10 min at room temperature and quenched with 50 mM NH₄Cl for 15 min at room temperature. After that, cells were blocked and permeabilized as described above. DAPI staining was performed (1:1000) for 5 min at room temperature. Samples were imaged with LSM780.

### Results

Silencing SORLA or Interfering with HER2 Signaling Leads to Dysfunctional Lysosomes

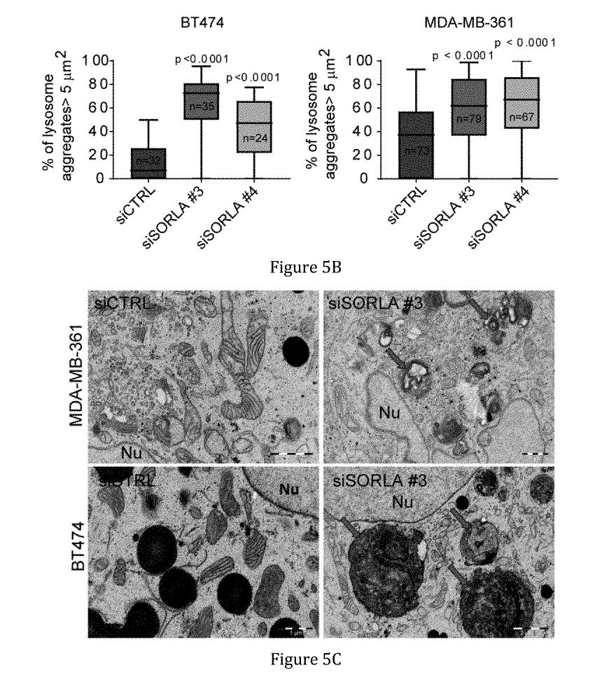

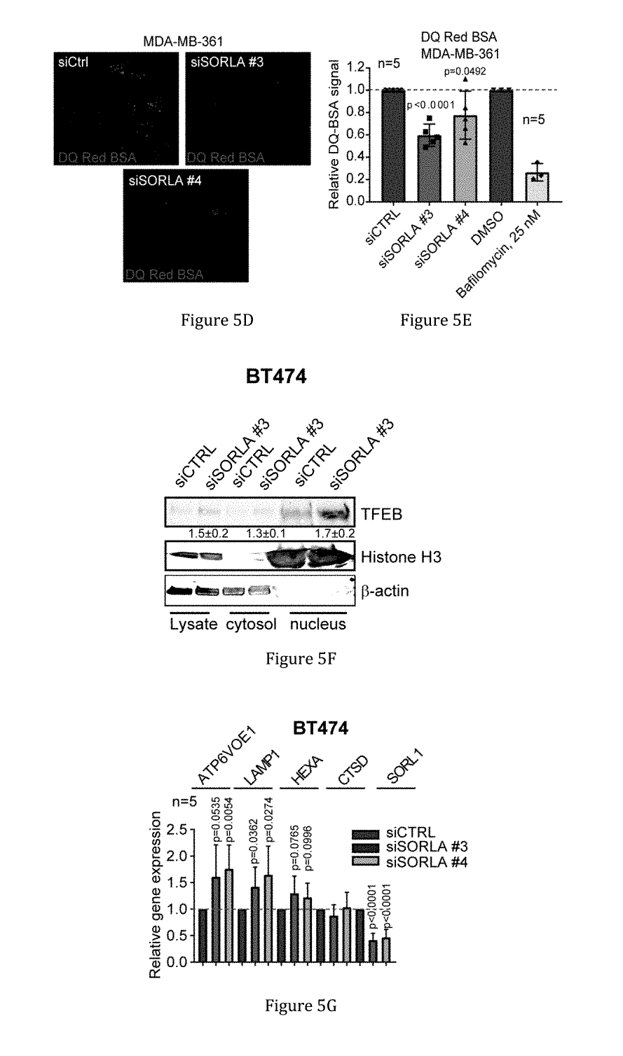

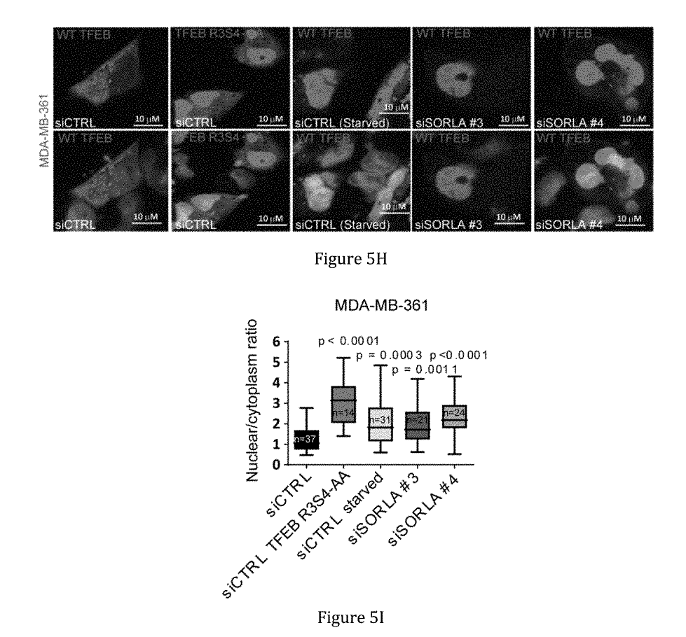

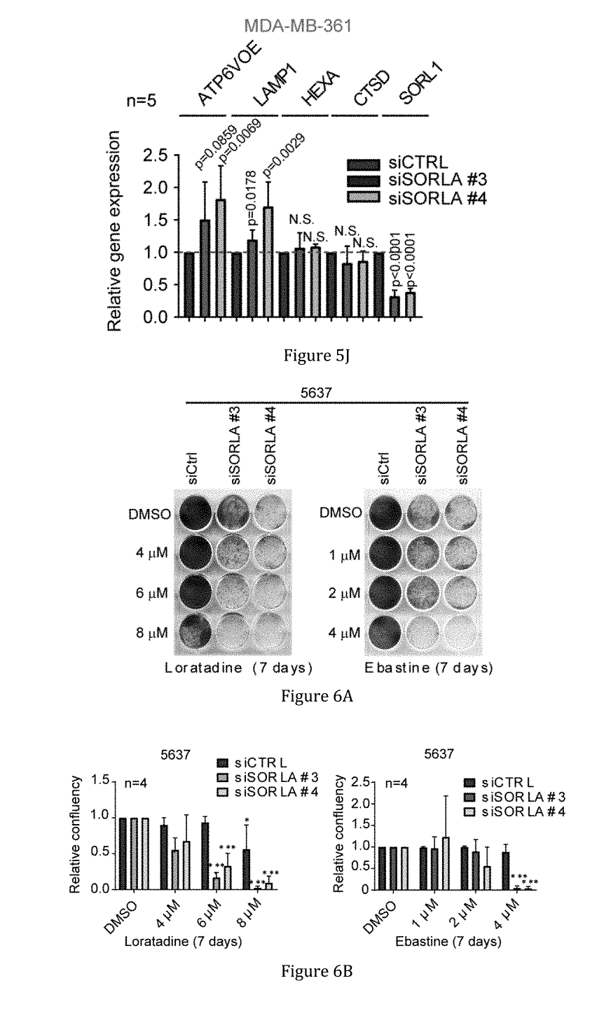

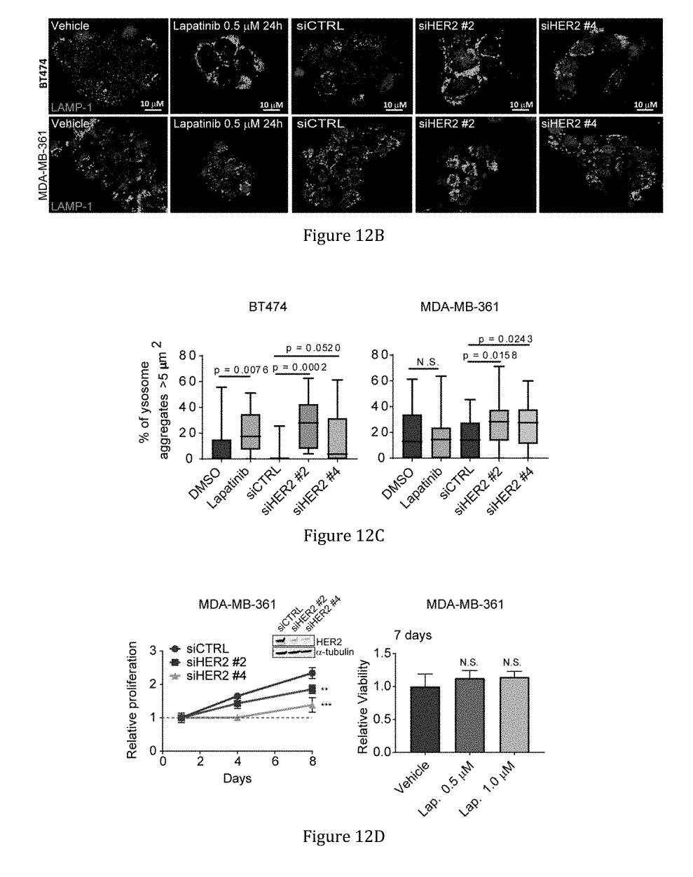

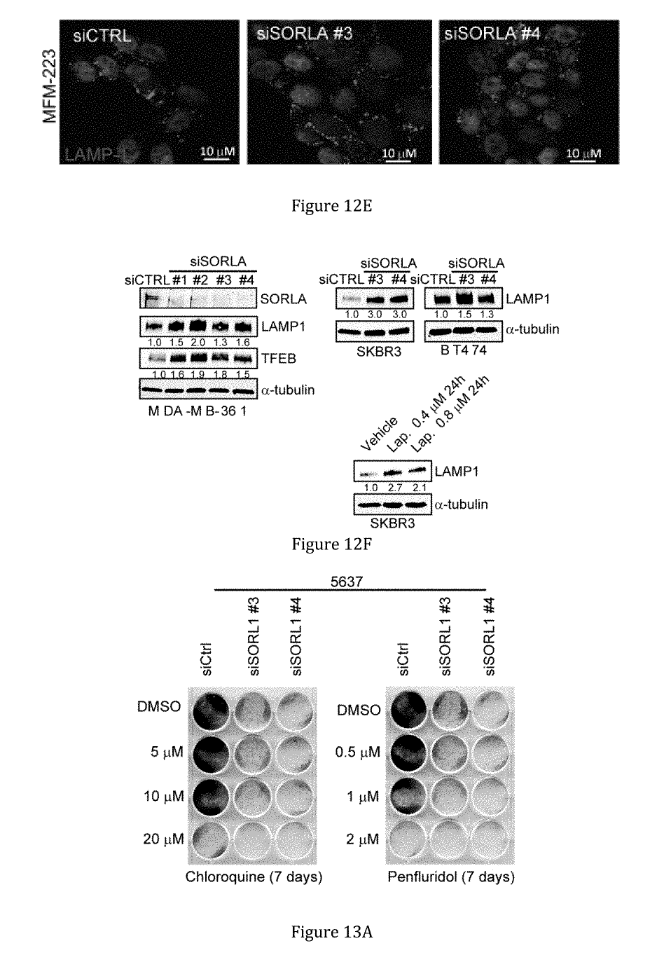

Treatment with GA triggers HER2 traffic to late endosomes resulting in rapid lysosomal degradation of the receptor (Cortese et al., 2013; Marx et al., 2010). The apparent discord between HER2 localization to late endosomes/lysosomes and the rather modest downregulation of HER2 protein levels in SORLA-silenced cells (about 50% in BT474, FIG. 4B) together with the enlarged lysosome structures evident in LAMP1 staining (FIG. 4J) could be indicative of compromised lysosome function. Accordingly, SORLA-silenced cells displayed strong perinuclear accumulation of LAMP1- and CD63 (LAMP3)-positive lysosomes (FIG. 5A) which were significantly more aggregated compared to control cells (FIG. 5B). The lysosomal aggregation was evident with four different siRNAs targeting SORLA (FIG. 12A). More detailed analyses using transmission electron microscopy (TEM) revealed enlarged lysosomes in SORLA-silenced cells indicative of a potential lysosome maturation defect (FIG. 5C). To monitor the proteolytic activity of lysosomes, we used DQ Red BSA staining, where the increase in DQ Red BSA signal has been linked to lysosomal proteolytic activity (Vazquez and Colombo, 2009). SORLA-silenced MDA-MB-361 cells showed significantly lower DQ Red BSA signal, detected either by confocal microscopy imaging (FIG. 5D) or by flow cytometry, than the respective control cells (FIG. 5E). Next we wanted to investigate if the lysosome phenotype is linked to decreased HER2 function. Silencing HER2 with two individual siRNAs triggered altered lysosomal localization similarly to SORLA silencing in BT474 and MDA-MB-361 cells (FIGS. 12B and 12C). In addition, lapatinib treatment induced lysosome aggregation specifically in the sensitive BT474 but not in the resistant MDA-MB-361 cells (FIGS. 12B and 12C). MDA-MB-361 cells are resistant to Lapatinib, but still dependent on HER2 function indicated by decreased proliferation after HER2 silencing (FIG. 12D). Thus, the lysosome phenotype seems to be linked to impaired cell growth (FIG. 12D). Moreover, when SORLA was silenced in the non-HER2-amplified breast cancer cells, MFM-223, there was no effect on lysosomal distribution or localization (FIG. 12E). These data together indicate a link between HER2 signaling and lysosome integrity in HER2-driven breast cancer cells, which is in line with previous studies showing that HER2 transformation changes lysosome localization and that lapatinib treatment induces perinuclear accumulation of lysosomes (Brix et al., 2014; Rafn et al., 2012). However, the lysosomal aggregation triggered by HER2 inhibition was not as dramatic as after SORLA silencing leaving open the possibility that other HER2-independent factors may be involved.

Transcriptional Activation of TFEB after SORLA Silencing

Transcription factor EB (TFEB) is a master transcriptional regulator of lysosome-related genes (Sardiello et al., 2009). TFEB up-regulates expression of many lysosome-related genes, such as LAMP1, ATP6V0E1, HEXA, CTSD etc. (Sardiello et al., 2009), by translocating into the nucleus in response to starvation and inhibition of mTORC1 (Martina et al., 2012; Roczniak-Ferguson et al., 2012). Since we detect a decrease in AKT/mTOR signaling as well as perinuclear accumulation of lysosomes, we sought to study the function of TFEB in more detail. Interestingly, silencing of SORLA in MDA-MB-361 cells with four distinct siRNAs induced a modest up-regulation of TFEB and LAMP1 protein levels when compared to control (FIG. 12F). Moreover, LAMP1 protein levels were similarly increased in SKBR3 and BT474 cells after SORLA silencing (FIG. 12F) and upon lapatinib treatment in SKBR3 cells (FIG. 12F). Since LAMP1 is a downstream target of TFEB and nuclear TFEB levels correlate with its transcriptional activity we investigated the effect of SORLA silencing on TFEB localization in cells. First we performed nuclear and cytoplasmic fractionation in control-silenced and SORLA-silenced BT474 cells. Again a modest up-regulation of TFEB was evident in lysates (1.5±0.2 folds) and in the cytosolic fraction (1.3±0.1 folds), but TFEB levels were clearly elevated in the nuclear fraction after SORLA silencing (1.7±0.2 fold, FIG. 5F). To further investigate TFEB activity, we analyzed gene expression of TFEB target genes by quantitative real time PCR in control-silenced and SORLA-silenced BT474 cells. Out of the four established TFEB target genes analyzed, we noticed up-regulation in three of the genes, namely LAMP1, ATP6VOE and HEXA (FIG. 5G). Next we used a TFEB reporter system to monitor the localization of TFEB after SORLA silencing or starvation in MDA-MB-361 cells. In line with previous reports wild-type TFEB-EGFP localized mainly to the cytoplasm in cells cultured in full medium and underwent nuclear translocation in response to starvation (FIGS. 5H and 5I). The positive control, phospho-deficient TFEB R3S4-AA-mutant with persistent nuclear localization, remained nuclear even in full medium, validating the functionality of the pathway in these cells (FIGS. 5H and 5I). Interestingly, silencing of SORLA led to increased nuclear localization of wild-type TFEB in cells growing in full medium, mimicking the effect of starvation of control cells (FIGS. 5H and 5I). Also in MDA-MB-361 cells, together with increased TFEB nuclear localization, we observed up-regulation of ATP6VOE and LAMP1 gene expression, but not HEXA and CTSD (FIG. 5J). These findings indicate that SORLA silencing, resulting in impaired HER2 function, has broad implications on the proliferation and fitness of HER2-driven cancer cells.

### Example 6

### SORLA Silencing and CAD Treatment has Additive Effect on Cell Viability of HER2-Dependent Cells

### Materials and Methods

Colony Formation Assay of SORLA Silenced 5637 Treated with CADs

Transient SORLA (with two individual siRNAs) or scramble silencing was performed for 5637 bladder cancer cells. After 48 hours of silencing, 20 000 cells per well were plated on colony formation assay on 12-well plate. Next day, cells were treated with increasing concentration of Ebastine (Sigma, E9531), Loratadine (Sigma, L9664), Penfluridol (Sigma, P3371) and Choloroquine (Biofellows, 4109). DMSO was used as control. Cells were treated for 7 days and drugs including media was replenished between 3-4 days of culture. After 7 days, cells were stained with 0.2% Crystal Violet in 10% EtOH for 10 min RT. Cells were washed extensively with PBS and dried. Wells were scanned and confluency was measured by using Image J colony area plug-in.

### Toxicity Curves of Ebastine in SORLA and Scramble Silenced Breast Cancer Cell Lines

Transient SORLA (with two individual siRNAs) or scramble silencing was performed for 5637 bladder cancer cells. After 48 hours of silencing, 3000 cells per well were plated on 96-well plate. Cells were treated with increasing concentration of Ebastine and Loratadine for 48 hours. After 48 hours of treatment, WST-8 reagent (Sigma, 96992) was added 10 μl/well and absorbance 450 nm was measured by plate reader after 1-2 h of incubation at 37° C. with 5% CO₂. Medium without cells was used as background and the A450 of background was subtracted from the samples. Relative viability was calculated by normalizing the A450 values of drug treated to DMSO treated cells. IC50 values were calculated by GraphPad using nonlinear regression to fit the data to the log(inhibitor) vs. response (variable slope) curve. IC50 values are represented as mean of three independent experiments.

### Results

Combined Depletion of SORLA and Treatment with Lysosome-Targeting CADs Promote a Synergistic Decrease in Cell Viability and Tumorigenesis in HER2-Driven Cancer Cells

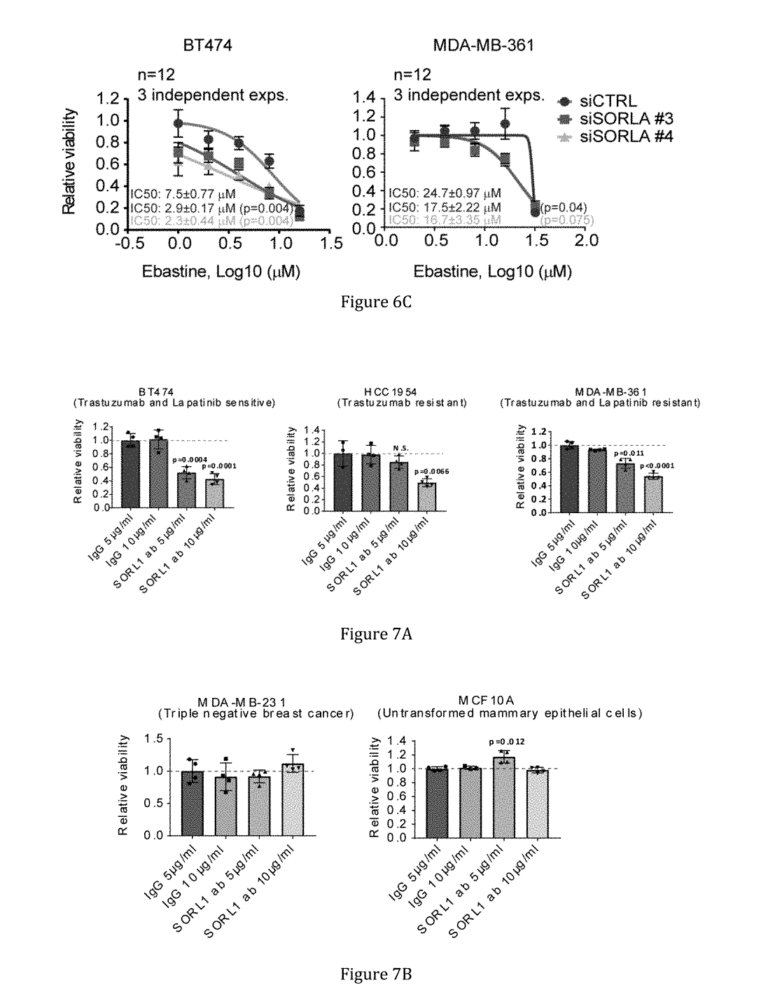

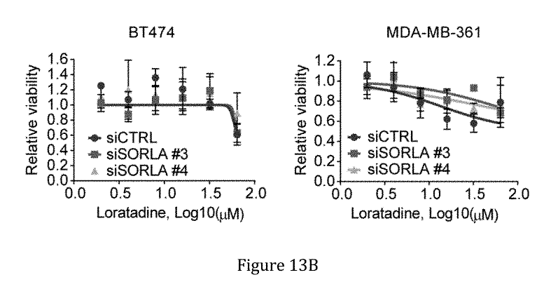

Previous studies indicate that cancer cells possess leaky lysosomes making them more susceptible to cationic amphiphilic drugs (CADs), a heterogeneous class of molecules with similar chemical structure resulting in lysosomal accumulation and inhibition (Petersen et al., 2013). Recently, cancer cells were shown to be more sensitive to CAD-induced cell death than un-transformed cells (Ellegaard et al., 2016). Given the defective lysosome phenotype of SORLA depleted cells, we were interested to these cells were sensitive to different classes of CADs. Out of four compounds tested (Penfluridol [anti-depressant], Loratadine [antihistamine], Ebastine [antihistamine] and lysosome and autophagy inhibitor choloroquine), the two antihistamines, Loratadine and Ebastine, demonstrated significant synergistic effects in ablating 5637 bladder cancer colony growth when combined with SORLA silencing (FIG. 6A, FIG. 6B and FIG. 13A). Ebastine also significantly decreased cell viability in combination with SORLA silencing in HER2-amplified breast cancer cells irrespective of their anti-HER2 therapy sensitivity (FIG. 6C and FIG. 13B). Our in vivo data indicate that silencing SORLA decreases proliferation without significant induction of apoptosis. However, combining SORLA silencing with Ebastine significantly increased cell death in vitro. Thus, SORLA-silenced cells show increased sensitivity to CADs, presumably due to silencing-induced lysosomal dysfunction.

### Example 6

Targeting SORLA with Monoclonal Antibody Inhibits Proliferation of HER2

### Materials and Methods

### Toxicity of SORLA Targeting Antibody and Trastuzumab Treated Breast Cancer Cells

Cells were plated on 96-well plate (3000 cells per well) in a volume of 100 μl. On next day, cells were treated with SORLA targeting monoclonal antibody (BD Transduction Lab, 612633) or with IgG control (BD Transduction lab, 554645) or with trastuzmab (Roche). Cells were treated totally for 7 days and between the days 3-4, media containing the antibodies were replenished. After 7 days of treatment, WST-8 reagent (Sigma, 96992) was added 10 μl/well and absorbance 450 nm was measured by plate reader after 1-2 h of incubation at 37° C. with 5% CO₂. Medium without cells was used as background and the A450 of background was subtracted from the samples. Relative viability was calculated by normalizing the A450 values of SORLA antibody or trastzumab treated cells to IgG control treated.

### Results

Antibody Targeting of SORLA Extra-Cellular Domain Inhibits Cell Proliferation of HER2-Dependent Cells and Synergizes with Trastuzumab

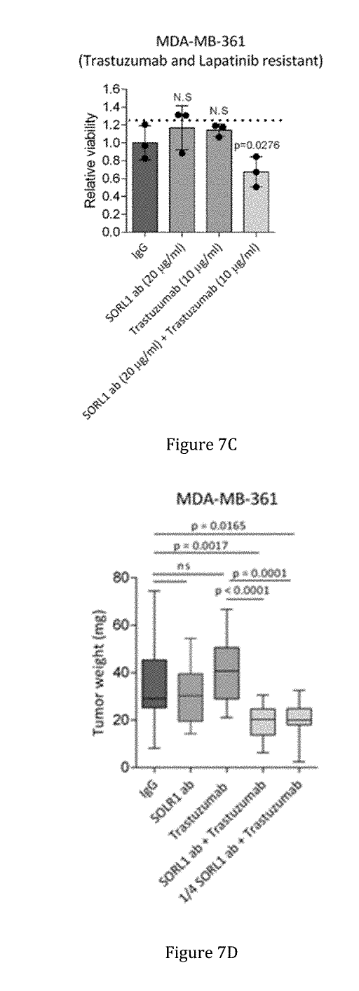

Our data indicate that SORLA associates and co-traffics with HER2 and loss of this complex dramatically impairs HER2 function in cancer cells. We hypothesized that antibody targeting the cell surface accessible SORLA ECD could potentially interfere with the SORLA-HER2 axis. To this end we employed a monoclonal antibody engineered to target the CR-cluster in the middle of SORLA ECD. SORLA antibody significantly inhibited proliferation of HER2-amplified breast cancer cells in vitro (FIG. 7A). Both anti-HER2 sensitive and resistance cell lines were sensitive to anti-SORLA treatment in vitro (FIG. 7A). However, non-HER2 and SORLA low cells, such as MDA-MB-231 and MCF10A, did not show decreased proliferation after SORLA antibody treatment (FIG. 7B) indicating the specificity of antibody to interfere the growth of HER2 amplified and SORLA expressing cells. Notably, SORLA-binding antibody sensitizes anti-HER2 therapy (Trastuzumab) resistant cells to therapy both in vitro and in vivo (FIGS. 7C and 7D). These preliminary results highlight the benefit of further development of functional blocking SORLA antibodies to potentially be applicable in treatment of HER2 dependent cancers.

### Example 7

In vitro validation of the most potent gRNAs:

Stable CAS9 expressing MDA-MB-361 cells are transfected with different gRNAs targeting SORLA gene. After transfection, cells are allowed to grow for 3-4 days and SORLA protein levels are detected from the pool of the cells by western blot. In addition, part of the cells are plated on 96-well plate as a single clones and gene editing of SORLA is detected from the clones by using surveyor assay and sequencing. This however, might be unfeasible for generation of stable SORLA knockdown cell lines, since the SORLA silencing and knockdown inhibits cell proliferation.

### Example 8

In vivo validation of the CRISPR-CAS9 mediated targeting of SORLA targeting:

MDA-MB-361 cells (5 million per mouse) are injected subcutaneously at the flank of 6 week old nude mice. After tumor formation (tumor volume of 100 mm3), SORLA targeting gRNA cloned into CAS9 adeno associated virus (AAV) vector is injected directly into tumor. Tumor volume is monitored for the following 3-4 weeks. After the 3-4 weeks of tumor growth, mice are sacrificed and some of the tumors are collected for western blot analysis to detect the efficiency of SORLA knockdown and some of the tumors are fixed in 10% formalin and processed for paraffin section with standard protocols. Immunohistological staining of SORLA is performed from the paraffin sections to confirm the SORLA knockdown efficiency.

It will be obvious to a person skilled in the art that, as technology advances, the inventive concept can be implemented in various ways. The invention and its embodiments are not limited to the examples described above but may vary within the scope of the claims.

1. A composition comprising:
a SORL1 inhibiting agent, selected for treating HER2-dependent cancer; and
a drug selected from the group consisting of cationic amphiphilic drugs (CAD) and HER2-targeted drugs.

2. The agent according to claim 1, wherein said agent inhibits SORL1 gene expression.

3. The agent according to claim 2, wherein said agent is selected from the group consisting of siRNA molecules, shRNA molecules, DsiRNA molecules, artificial miRNA precursors, and antisense oligonucleotides.

4. The agent according to claim 3, wherein said agent is siRNA comprising:
a sequence selected from the group consisting of SEQ ID Nos: 1-18.

5. The agent according to claim 3, wherein said agent is shRNA comprising:
a sequence selected from the group consisting of SEQ ID Nos: 19 and 20.

6. The agent according to claim 1, wherein said agent is a gene editing agent.

7. The agent according to claim 6, wherein said agent comprises:
a polynucleotide encoding CRISPR associated protein 9 (Cas9); and
a guide nucleotide sequence directing Cas9 nuclease to an SORL1 gene region.

8. The agent according to claim 7, wherein said guide nucleotide sequence comprises:
a sequence selected from the group consisting of SEQ ID Nos: 21-51.

9. The agent according to claim 1, wherein said agent is an antibody.

10. The agent according to claim 9, wherein said antibody binds to an SORL1 extracellular domain and disrupts association between SORL1 and HER2.

11. The agent according to claim 9, wherein said antibody is a dual-specificity antibody binding simultaneously to SORL1 and HER2.

12. The agent according to claim 1, wherein said cancer is selected from the group consisting of breast cancer, ovarian cancer, gastric cancer, colorectal cancer, bladder cancer and any cancer that is HER2-dependent.

13. (canceled)

14. (canceled)

15. Method for treating HER2-dependent cancer, the method comprising:
administering a therapeutically effective amount of an SORL1 inhibiting agent to a subject in need thereof.

16. The method according to claim 15, wherein said agent inhibits SORL1 gene expression.

17. The method according to claim 16, wherein said agent is selected from the group consisting of siRNA molecules, shRNA molecules, DsiRNA molecules, artificial miRNA precursors, and antisense oligonucleotides.

18. The method according to claim 17, wherein said agent is siRNA comprising:
a sequence selected from the group consisting of SEQ ID Nos: 1-18.

19. The method according to claim 15, wherein said agent is a gene editing agent.

20. The method according to claim 19, wherein said agent comprises:
a polynucleotide encoding CRISPR associated protein 9 (Cas9); and
a guide nucleotide sequence directing Cas9 nuclease to an SORL1 gene reg
